(19) United States
(12) Patent Application Publication
(10) Pub. No.: US 2026/0263373 A1
(43) Pub. Date: Sep. 10, 2026

(54) AMPHIPHILIC NANOPARTICLES TO TARGET AMYLOID BETA-INDUCED MICROGLIAL ACTIVATION

(71) Applicants: Rutgers, The State University of New Jersey, New Brunswick, NJ (US); The Regents of the University of California, Oakland, CA (US)

(72) Inventors: Hoda M. Gebril, Monroe Township, NJ (US); Prabhas V. Moghe, Dallas, TX (US); Kathryn E. Uhrich, Riverside, CA (US)

(73) Assignees: Rutgers, The State University of New Jersey, New Brunswick, NJ (US); The Regents of the University of California, Oakland, CA (US)

(21) Appl. No.: 19/662,865

(22) Filed: Apr. 29, 2026

Related U.S. Application Data
(63) Continuation-in-part of application No. PCT/US2024/053545, filed on Oct. 30, 2024.
(60) Provisional application No. 63/594,796, filed on Oct. 31, 2023.

(51) Int. Cl. A61K 9/51; A61K 9/00; A61K 31/77; A61P 25/28

(52) U.S. Cl. CPC A61K 9/5138; A61K 9/0019; A61K 9/5192; A61K 31/77; A61P 25/28

(57) ABSTRACT

Nanoparticle compositions and methods inhibiting amyloid beta (Aβ) fibrilization in a subject in need thereof using nanoparticles to target fibril β-amyloid (fAβ)-specific scavenger receptors. Also provided is a method of using nanoparticle compositions to treat Alzheimer's disease (AD) and similar amyloidopathies.

### CROSS-REFERENCE TO RELATED APPLICATIONS

This application is a continuation-in-part of and claims the benefit under 35 U.S.C. § 120 of the earlier filing date of International Patent Application No. PCT/US2024/053545, filed on Oct. 30, 2024, which, in turn, claims the benefit under 35 U.S.C. § 119(e) of the earlier filing date of U.S. Provisional Application No. 63/594,796, filed on Oct. 31, 2023. Each disclosure is incorporated herein by reference in its entirety.

### STATEMENT REGARDING FEDERALLY SPONSORED RESEARCH OR DEVELOPMENT

This invention was made with government support under grant number AG060024 awarded by the National Institutes of Health and grant number 1803675 awarded by National Science Foundation. The government has certain rights in the invention.

### FIELD OF THE INVENTION

This invention relates to nanoparticle compositions and inhibiting amyloid beta (Aβ) fibrilization in a subject in need thereof using nanoparticles that target fibril β-amyloid (fAβ)-specific scavenger receptors. The invention also relates to a method of treating Alzheimer's disease (AD) and similar amyloidopathies using nanoparticles.

### BACKGROUND OF THE INVENTION

Alzheimer's disease (AD) is the sixth leading cause of death among Americans, with an estimated 5.8 million individuals of age 65 and older with AD. As AD accounts for an estimated 60% to 80% of the dementia cases, the socio-economic burden of the disease has grown by 35% since 2010. The global cost of the disease is estimated to reach two trillion dollars by 2030. Early clinical stages of AD are associated with profound neuroinflammation, intracellular neurofibrillary tangles, and extracellular deposits of amyloid plaques of fibril β-amyloid (fAβ) that are generated via the proteolytic cleavage of amyloid precursor protein (APP) by β0 and γ secretases. While approximately 5% of AD cases are characterized by early onset of symptoms due to inheritable genetic mutations, the vast majority of AD cases are multi-factorial in etiology and characterized by sporadic late onset. Nevertheless, the single risk factor for sporadic AD is age. This presents an immense health challenge, especially with the increasing longevity of the world's growing population.

In AD patients, Aβ exists in soluble oligomeric forms and insoluble fibrils in the parenchymal extracellular space. fAβ drives several cytopathological changes, including activation of glial cells such as microglia and astrocytes, which ultimately leads to irreversible neuronal damage. Currently, the non-curative FDA-approved AD drugs are used only to either delay clinical decline in AD individuals or temporarily mitigate symptoms due to mild to moderate AD. Under the very recent accelerated approval pathway, the only curative FDA-approved drug currently available was designed to remove fAβ deposits in early stages of AD. Although this supports the hypothesis that fAβ is an early event in AD, the disease pathogenesis and etiology remain diverse and multifactorial because of the inclusion of other events such as aberrant tau accumulation and neuroinflammation.

Microglia are the primary immune cells of the central nervous system (CNS), accounting for approximately 5-15% of cells in the CNS. They play a pivotal role in brain homeostasis, including neuronal maintenance, immune surveillance, and clearance of misfolded proteins, pathogens, and cell debris. In AD, resident resting microglia are activated and migrate to regions of dense fAβ plaques to promote fAβ clearance. However, chronic activation of microglia leads to the release of pro-inflammatory cytokines and chemokines, which exacerbates the progression of the disease. A growing body of evidence suggests that functional and healthy microglia can efficiently restrict fAβ pathological overgrowth. In line with this notion, activated microglia have been linked to Aβ seeding and plaque growth. Moreover, recent studies suggest that aging is associated with dysfunctional microglia. Therefore, microglia can serve as a potential therapeutic target for age-related disorders, including AD.

The physical association of microglia with fAβ in AD suggests the involvement of surface receptors in this interaction. Scavenger receptors (SRs) are structurally conserved membrane receptors highly expressed in macrophages, microglia, and endothelial cells. To date, several SRs have been associated with the pathogenesis of fAβ. Class A (SRA-1) and B (CD36) SRs have been associated with the internalization of fAβ and the subsequent inflammatory response. Other receptors associated with fAβ pathogenesis are CD14, TLR2, and TLR4. The inhibition of CD36-fAβ interaction was found to halt fAβ-mediated microglial immune response. Moreover, in vivo studies in CD36 deficient mice reported reduced fAβ-mediated oxidative stress and microglial recruitment. Importantly, the SRA1 deficiency as well as complete knockout resulted in a significant reduction in fAβ microglial uptake. Other SRs such as Class D SR, CD68, which is expressed on lysosomes and endosomes of microglia, were overexpressed in human AD, however, their role in fAβ trafficking has not been systematically elucidated. The strong effect of SRs on modulating fAβ-mediated pathology makes them potential candidates for therapeutic applications.

Research on synthetic compounds that possess the physical properties of SR-binding ligands has been reported. Approaches targeting CD36 have been explored as a means to ameliorate neuroinflammation associated with neurodegenerative disorders. While small molecule ligands to block interactions between CD36 and fAβ have shown promising results, their low solubility in water, their short half-life, and low bioavailability limit their wide application in the pharmaceutical field. Advances in nanomedicine have ushered in new classes of therapeutics against CNS disorders utilizing features such as high biocompatibility, bioavailability, and structural stability to ensure targeted delivery. Nanoparticles and nanocarrier-based approaches have been used to target fAβ-mediated pathology. Notably, most of these approaches were designed to target one aspect of disease pathology, but with limited ability to address the mechanistic triggers for the disease. Thus, there remains a need to develop therapies to treat AD that target the underlying causes.

### SUMMARY OF THE INVENTION

The present invention relates to inhibiting fAβ-mediated pathology with nanoparticles as well as using nanoparticles to treat a neurodegenerative disease.

One aspect of the present invention provides a method of inhibiting amyloid beta (Aβ) fibrilization in a subject in need thereof, comprising administering a nanoparticle to the subject, wherein the nanoparticle comprises a hydrophobic core and a bioactive amphiphilic macromolecule that forms a shell coating the hydrophobic core, and wherein the bioactive amphiphilic macromolecule comprises a structure set forth in Formula A or Formula B:

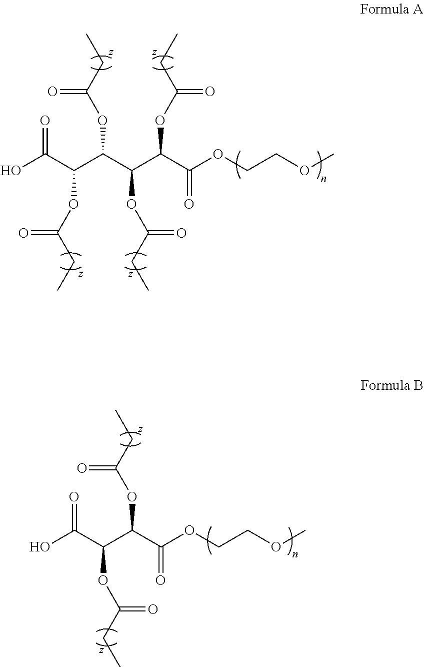

wherein n is an integer from 100 to 125; and
wherein z is an integer from 2 to 20.

In some embodiments, z is an integer from 4 to 12.

In one embodiment, z is 10. In some embodiments, n is an integer from 110 to 120.

In some embodiments, the diameter of the nanoparticle ranges from 90 nm to 200 nm. In one embodiment, the diameter of the nanoparticle is 90 nm to 160 nm.

In one embodiment, the polydispersity index (PDI) of the nanoparticle is less than 0.3.

In one embodiment, the bioactive amphiphilic macromolecule to hydrophobic core ratio by weight is 8:2.

In one embodiment, the nanoparticle binds to a fibril β-amyloid (fAβ)-specific scavenger receptor. In some embodiments, the fAβ-specific scavenger receptor is selected from the group consisting of CD36, CD68, and SRA1.

In one embodiment, the hydrophobic core comprises a structure set forth in Formula C:

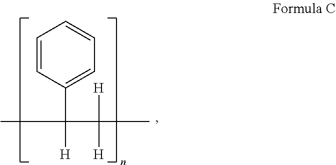

wherein n is an integer from 10 to 120. In some embodiments, m is an integer from 10 to 15.

In one embodiment, the bioactive amphiphilic macromolecule consists of the structure set forth in Formula A:

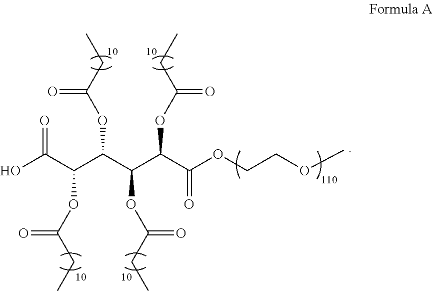

In one embodiment, the bioactive amphiphilic macromolecule consists of the structure set forth in Formula B:

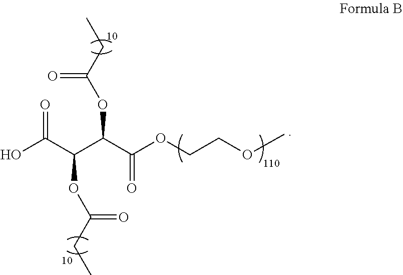

In one embodiment, the subject is a human.

In some embodiments, wherein the nanoparticle:
a) suppresses Aβ-induced microglial activation;
b) reduces aggregation of Aβ; and/or
c) promotes fAβ clearance;

In one aspect of the present invention, provided is a method of treating Alzheimer's disease (AD) in a subject in need thereof, comprising administering a nanoparticle to the subject, wherein the nanoparticle comprises a hydrophobic core and a bioactive amphiphilic macromolecule coating the core, and wherein the bioactive amphiphilic macromolecule comprises a structure set forth in Formula A or Formula B:

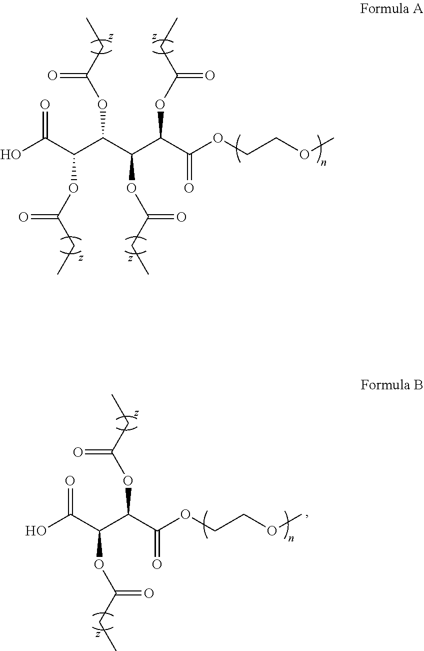

wherein n is an integer from 100 to 125; and
wherein z is an integer from 2 to 20.

In some embodiments, the nanoparticle:
a) blocks Aβ fibrilization;
b) promotes fAβ clearance;
c) reduces internalization of Aβ fibrilization; and/or
d) suppresses Aβ-induced microglial activation.

In some embodiments, the Alzheimer's disease is early-stage Alzheimer's disease, moderate Alzheimer's disease, or late-stage Alzheimer's disease.

In some embodiments, the nanoparticle modulates active microglia-mediated neurotoxicity.

In some embodiments, the nanoparticle reduces levels of pro-inflammatory cytokines and chemokines.

In some embodiments, the subject has previously received treatment for Alzheimer's disease with an existing therapeutic agent.

### BRIEF DESCRIPTION OF THE DRAWINGS

FIGS. 1A-1D. Nanoparticle fabrication via flash nanoprecipitation. FIG. 1A is a schematic diagram of the flash nanoprecipitation (FNP) procedure for the synthesis of AM-nanoparticles (NPs). A confined impinging jet mixer (CIJ) was used to mix a stream of 250 μL of 50/50% (v/v) mixture of tetrahydrofuran (THF) containing 8 mg/ml shell molecule, and 2.5 mg/ml hydrophobic core molecule with 250 μL of an aqueous stream. Key element of FNP technique for the fabrication of stable NPs is the induction time of aggregation and precipitation. The time required for a complete and homogenous mixing of the aqueous and solvent stream is Tmix, while the precipitation time of NPs is Tflash. FIG. 1B is a table listing chemical structures of AM shell molecules and polystyrene core. FIG. 1C depicts transmission electron microscope (TEM) images of NPs. FIG. 1D is a table that summarizes characterization of NPs radius and polydispersity index (PDI). Data are presented as mean SEM; n=3.

FIGS. 2A-2D. Development of fibril Amyloid beta (fAβ) and effect of NPs on amyloid beta fibrilization kinetics. FIG. 2A shows representative TEM images of fibril Aβ (fAβ) and oligomeric Aβ (oAβ). 5 μL of fAβ or oAβ was loaded on a formvar-coated, carbon-stabilized copper grid. Scale bar is 200 nm. FIG. 2B is a graph of the results of a Thioflavin-T (Th-T) assay for fAβ and oAβ to validate aggregation of β sheets. Protein or PBS (control) was mixed with 40 μM Th-T in a black bottom 96 well microplate. Data are presented as mean SEM; n=3; **p=0.003 for student-t test. FIG. 2C is a graph of the results of a Thioflavin T kinetic assay conducted with Aβ monomer over 65 h at 37° C. Samples containing NPs or monomeric Aβ only or in the presence of NPs at a 1:10 volume ratio were loaded with 20 μM Th-T into 96 well clear bottomed non-binding half-area plates. FIG. 2D is a graph of endpoint Th-T fluorescence for Aβ combination with NPs at 63 h. Data are presented as mean±SEM; n=β-4; ****p<0.0001 for T₁₂P₅(PS) vs fAβ488 and ***p=0.0007 for M₁₂P₅(PS) vs fAβ488 for Dunnett's multiple comparisons shown on graph by One-Way ANOVA.

FIGS. 3A-3D. Competitive receptor binding assay in vitro. NPs reduce surface active sites of CD36, CD68, and SRA1 on the surface of BV-2 microglia. FIG. 3A is a graph of activity for the surface active sites of CD36. FIG. 3B is a graph of activity for the surface active sites of CD68. FIG. 3C is a graph of activity for the surface active sites of SRA1. FIG. 3D is a graph of activity for the surface active sites of TLR2. BV2 microglia were incubated with NPs for 1 h, then co-incubated with SR-specific antibody or isotype controls for 30 min. Data are presented as mean±SEM; n=3; ***p=0.0001 for A, **p=0.009 for B, **p=0.0048 for C, for Dunnett's multiple comparisons shown on graph by One-Way ANOVA.

FIG. 4. Scavenger receptors interrupt fibril amyloid beta (fAβ) microglial internalization. BV2 microglia treated with antibodies against scavenger receptors CD36, CD68, TLR2, and SRA1 or isotype control for 24 h then co-incubated with Alexa fluor 488-labeled fAβ for 24 h. Quantitative measurement of the intracellular Aβ488 in BV2 cells. Data are presented as mean±SEM; n=4; ****p<0.0001 for Dunnett's multiple comparisons shown on graph by One-Way ANOVA.

FIGS. 5A-5D. Effect of NPs on amyloid beta (Aβ) cellular uptake. FIG. 5A shows confocal images of intracellular Alexa fluor 488-labeled fAβ and brightfield illuminated cells. Scale bar is 50 μm. BV2 microglia treated with nanoparticles for 24 h then co-incubated with Alexa fluor 488-labeled fAβ for 24 h. FIG. 5B depicts quantitative measurement of the intracellular fluorescence of Aβ488 in BV2 microglia. Data are presented as mean±SEM; n=4; **p=0.0016 for T₁₂P₅(PS) vs fAβ488, and **p=0.0059 for M₁₂P₅(PS) vs fAβ488, for Dunnett's multiple comparisons shown on graph by One-Way ANOVA. FIG. 5C shows images of Thioflavin-S stain of fAβ in BV2 microglia (brightfield) treated with NPs for 24 h then co-incubated with fAβ for 24 h. FIG. 5D depicts quantitative measurement of the intracellular fluorescence thioflavin-S stain in BV2 microglia. Data are presented as mean SEM; n=4; ****p<0.0001 for T₁₂P₅(PS) vs fAβ488, and ****p<0.0001 for M₁₂P₅(PS) vs fAβ3488, for Dunnett's multiple comparisons shown on graph by One-Way ANOVA.

FIGS. 6A-6D. AM-NPs suppress Aβ-mediated microglial activation. FIG. 6A depicts quantitative analysis of fluorescence intensity of iNOs in BV2 cells after 24 h co-incubation with fAβ and NPs. Data are presented as mean±SEM; n=3; ***p=0.0002 for T₁₂P₅ vs fAβ, ***p=0.0001 for M₁₂P₅ vs fAβ for Dunnett's multiple comparisons shown on graph by One-Way ANOVA. FIG. 6B is a graph of nitrite concentration measured by Griess reagent in the conditioned media harvested 24 h after cells were co-incubated with Aβ and NPs. Data are presented as mean±SEM; n=3; **p=0.0057 for T₁₂P₅ vs fAβ3, **p=0.0048 for M₁₂P₅ vs fAβ for Dunnett's multiple comparisons shown on graph by One-Way ANOVA. FIG. 6C is a graph of the concentration of TNFα measured by ELISA in the conditioned media harvested 24 h after cells were co-incubated with fAβ and NPs. Data are presented as mean SEM; n=3; **p=0.042 for T₁₂P₅ vs fAβ, **p=0.0068 for M₁₂P₅ vs fAβ for Dunnett's multiple comparisons shown on graph by One-Way ANOVA. FIG. 6D is a graph of the % microglia-induced neurotoxicity in SH-SY5Y cells that were treated with media conditioned by fAβ or LPS-stimulated BV2 cells in the presence or absence of NPs. Cytotoxicity in response to stimulated BV2 conditioned media was quantified in SH-SY5Y media using LDH assay. Data are presented as mean±SEM; n=3; ***p=0.003 for T₁₂P₅ vs fAβ and **p=0.0027 for M₁₂P₅ vs fAβ for Dunnett's multiple comparisons shown on graph by One-Way ANOVA.

FIGS. 7A-7B. NPs mediate fAβ-induced NF-κB nuclear translocation in microglia. FIG. 7A shows representative confocal microscopy images showing NF-κB nuclear translocation. BV2 microglia were treated with 20 μM fAβ with or without NP for 2 h. Scale bar is 25 μm. FIG. 7B shows quantitative measurement of the nuclear translocation of NF-κB in BV2 cells treated with Aβ with or without NPs for 2 h. Data are presented as mean±SEM; n=3; **p=0.0027 for fAβ vs control, **p=0.0044 for T₁₂P₅ vs fAβ and **p=0.0054 for M₁₂P₅ vs fAβ, for Dunnett's multiple comparisons shown on graph by One-Way ANOVA.

FIGS. 8A-8F. AM-NPs accelerate lysosomal clearance of fAβ. FIG. 8A shows representative confocal microscopy images showing fAβ-positive lysosomes. White arrows point at fAβ in lysosomes. Scale bar is 25 μm. BV2 microglia were incubated with NPs for 24 h and then were co-incubated with Alexa Fluor 488-labeled fAβ for 2 h or 24 h. Lysosomes in treated live BV2 microglia were stained with 70 μM LysoTracker™ for 30 mi. Cells were then fixed using 4% PFA. FIG. 8B shows quantitative measurement of the lysosomal degradation of intracellular Aβ at 2 h and 24 h in BV2 microglia that were pretreated with NPs. Data are presented as mean±SEM; n=3; ****p<0.0001 for T₁₂P₅ vs fAβ, for Dunnett's multiple comparisons shown on graph by Two-Way ANOVA. FIG. 8C shows orthogonal projection of confocal Z-stacks that show co-labeling of CD68, Dil-labeled NPs, and nuclear stain Hoechst. BV2 microglia were incubated with NPs for 24 h and were then co-incubated with Alexa Fluor 488-labeled fAβ for 2 h. Cells were fixed with 4% PFA, permeabilized using PBS-T, and then blocked using 2% goat blocking buffer. Cells were then incubated with anti CD68, LAMP-1 antibodies overnight at 4° C., then washed with PBS-T. Cells were incubated with secondary antibody Alex 488 or 954 for 1 h and nuclei were counterstained with Hoechst. FIG. 8D shows orthogonal projection of confocal Z-stacks that show co-labeling of LAMP-1 (white), Dil-labeled NPs, nuclear stain Hoechst, and fAβ488 in brightfield illuminated cells in the top panel, and co-labeling of nuclear stain Hoechst, Dil-labeled NPs, and fAβ488 in brightfield illuminated cells in the bottom panel.

White arrows show intracellular fAβ3. E&F) BV2 microglia incubated with NPS for 24 h incubation then co-incubated with fAβ for 30 min. FIG. 8E shows confocal images of LC3 immunoreactivity in BV2 microglia that were co-stained with Hoechst. FIG. 8F depicts quantitative analysis of LC3 fluorescence intensity in BV2 microglia. Data are presented as mean±SEM; n=3; **p<0.0045 for T₁₂P₅ vs fAβ, for Dunnett's multiple comparisons shown on graph by Two-Way ANOVA. Scale bar is 25 μm.

FIGS. 9A-911. AM-NPs accelerate clearance of fAβ. FIGS. 9A-9D are electron microscope micrographs of BV2 microglia that were incubated with NPs for 24 h, and then were co-incubated with fAβ for 30 min. Cells were fixed with 2.5% glutaraldehyde 4% PFA in 0.1 M cacodylate buffer at pH 7.4. Ultrathin (90 nm) sections of cell pellets were imaged using the electron microscope. FIGS. 9A and 9E show the ultrastructure of a microglia treated with fAβ only. The intracellular fAβ (white arrowhead) is surrounded by damaged vacuoles, including large autophagosomes (white arrow) and lysosomes with incomplete membranes. Mitochondria (M) in this cell were structurally damaged, as evidenced by disordered cristae and a thin outer membrane (FIG. 9E). FIGS. 9B and 9F show the ultrastructure of a fAβ-treated microglia co-incubated with T₁₂P₅(PS). In this cell, the phagocytic activity was high, as illustrated by the high representation of a variety of autophagosomes (white arrow), lysosomes with well-developed membranes, and autophagosome-lysosome fusion structures (grey arrow). Mitochondria in this cell were intact, as evidenced by well-defined cristae structures and an intact outer membrane (FIG. 9F). FIGS. 9C and 9G show the ultrastructure of fAβ-treated microglia that were co-incubated with M₁₂P₅(PS). The phagocytic activity was less, as illustrated by fewer autophagosomes (white arrow); however, the lysosomal structures are characterized by a well-developed double membrane (yellow arrow). This cell exhibited high representation of intact and long mitochondria characterized by well-defined cristae and an intact outer membrane (FIG. 9G). FIGS. 9D and 9H show the ultrastructure of fAβ-treated microglia that were co-incubated with the control PS-b-PEG(PS). A large center of fAβ is present in this cell, along with large thin-walled autophagosomes (white arrow) and thin-walled lysosomal structures. Mitochondria in this cell were structurally damaged, as evidenced by distorted cristae and thin outer membrane. N, nucleus, and M, mitochondria. Scale bar is 500 nm in the top panel of FIGS. 9A-9H. In the bottom panel in FIGS. 9A-9D scale bar is 1 μm.

FIG. 10. is a schematic of AM-NPs' effects to interrupt amyloid beta fibrilization, cellular trafficking, and fAβ-mediated neuronal damage. 1. NPs interrupt Aβ fibrilization. 2a & 2b. NPs bind to the fAβ-binding scavenger receptors SRA1, CD36, and CD68 and interrupt fAβ microglial fAβ-binding. 3a. NPs induce lysosomal fAβ degradation. 3b NPs modulate NF-κB nuclear translocation. 4-6 AM-NPs modulate microglial inflammatory response and neurotoxicity.

FIGS. 11A and 11B illustrate the maximum tolerated dose of AM-NPs. FIG. 11A is a schematic that summarizes the study design in wild-type B6129SF2/J mice administered 0.4 mg/kg, 1.2 mg/kg, or 2 mg/kg of T₁₂P₅(PS). FIG. 11B is a graph that summarizes the body weight of the mice treated with 0.4 mg/kg, 1.2 mg/kg, or 2 mg/kg of T₁₂P₅(PS).

FIG. 12 is a schematic of a study to determine the efficacy of AM-NPs in 3XTG-AD mice treated with T₁₂P₅(PS) or control NPs (PS-b-PEG).

FIGS. 13A and 13B illustrate that T₁₂P₅ (PS+Dil) penetrates human cerebral organoids (hmCOs). FIG. 13A is a schematic overview of the method used to obtain images of hmCOs treated with T₁₂P₅(PS). FIG. 13B depicts images of hmCOs treated with 0.035 mg/ml, 0.14 mg/ml, or 0.35 mg/ml T₁₂P₅(PS). DAPI was used to stain the nucleus.

FIGS. 14A-14C illustrate that AM-NPs internalize the hCMEC/D monolayer cell layer of the human blood-brain barrier (BBB). FIG. 14A is a schematic diagram illustrating the monoculture system of hCMEC/D3 cells where the cells were seeded on the apical side of a transwell insert. AM-NPs were added to the apical side, and media was collected from the basolateral side over 24 and 48 hours. FIG. 14B. is a graph of the Dextran Permeability of the hCMEC/D3 Cell Line in Apical to Basolateral direction to measure the membrane efficacy and the tight junction formation. hCMEC/D3 cells were cultured in the presence of 70 kDa fluorescently labeled dextran (1 μM) in the apical chamber. Fluorescence was measured in the basolateral chamber at given time points. Barrier formation was observed for 70 kDa dextran (permeability coefficient=0.36*10-3 cm/s) when comparing empty inserts with inserts containing hCMEC/D3 cells. FIG. 14C. is a table of transendothelial electrical resistance (TEER) of hCMEC/D3 cells measured at the apical side at different time points (0, 24, 28 h), as an indicator of barrier integrity, showing sustained membrane tightness over time in the presence of AM-NPs.

FIG. 15 shows that AM-NPs undergo endocytosis into hCMEC/D3 cells. Quantitative analysis of intracellular Rh (rhodium)-AM-NPs at different time points. Data are presented as mean±SEM.

FIGS. 16A-16E illustrate AM-NPs transcytosis across the monolayer of hCMEC/D3 cells. FIGS. 16A-16D show quantitative measurement of AM-NPs transcytosis through hCMEC/D3 cells from apical (A) to basal (B) direction. Rhodamine-tagged NPs were added to the apical sides of inserts with (solid lines) or without cells (dashed lines). Then, the media was collected from A and B at different time points (1, 2, 24, and 48 hr), and rhodamine fluorescence was measured using a plate reader to calculate A-to-B transcytosis. FIG. 16A corresponds to T₁₂P₅(PS). FIG. 16B corresponds to M₁₂P₅(PS). FIG. 16C corresponds to PEG-b-PLA(PS). FIG. 16D corresponds to all nanoparticles (NPs). FIG. 16E shows quantitative analysis of the apparent permeability coefficient (Papp) for each NPs across hCMEC/D3 cells after 48 hr. Data are presented as mean±SEM. n=4; ****p<0.005 for T₁₂P₅(PS) vs PEG-b-PLA(PS) and *p=0.01 for M₁₂P₅(PS) vs PEG-b-PLA(PS) for Dunnett's multiple comparisons shown on graph by one-way ANOVA.

FIGS. 17A-17C show the ultrastructure of hCMEC/D3 monolayer at 7 days of in vitro culture and after 2 hours of AM-NPs treatment. FIG. 17A shows electron microscope micrographs of hCMEC/D3 cell layer illustrating the formation of tight junction (green arrow) with visible attachment to the membrane and exposure to the apical side of the insert. Cells were fixed with 2.5% glutaraldehyde 4% PFA in 0.1M cacodylate buffer at pH 7.4. Ultrathin (90 nm) sections of cell pellets were imaged using the electron microscope.

FIGS. 17B and 17C show AM-NPs uptake into the cells in endocytic vacuoles (lightest grey arrow) and the exocytosis of NPs (medium grey arrow) to the other side of the membrane (dark gray arrow). Scale bars are 500 nm in FIGS. 17A and 17B, and 100 nm in FIG. 17C.

FIG. 18 shows that AM NPs preserve neuronal integrity in 3xTg-AD mice. Representative fluorescent images showing neurofilament medium chain (NFM) immunoreactivity in the cerebral cortex (CTX) of 30 μm brain sections from 5.5-month-old 3xTg-AD mice intracranially injected with equivalent volumes of either T₁₂P₅(PS) NPs (2 mg/kg), PS-b-PEG(PS) control NPs, or saline. NFM staining indicates preserved neuronal cytoskeletal structure in T₁₂P₅(PS)-treated brains compared to control groups (saline and PS-b-PEG[PS]). Scale bar: 100 μm.

FIG. 19 shows that AM NPs preserve neuronal integrity in 3xTg-AD mice. Representative fluorescent images showing neurofilament medium chain (NFM) immunoreactivity in the cerebellum (CB) of 30 μm brain sections from 5.5-month-old 3xTg-AD mice intracranially injected with equivalent volumes of either T₁₂P₅(PS) NPs (2 mg/kg), PS-b-PEG(PS) control NPs, or saline. NFM staining indicates preserved neuronal cytoskeletal structure in T₁₂P₅(PS)-treated brains compared to control groups (saline and PS-b-PEG[PS]). Scale bar: 100 μm.

FIG. 20 shows that AM NPs attenuate demyelination in 3xTg-AD mice. Representative images of Black Gold II (BG) stain of myelin sheath in the cerebral cortex of 30 μm brain sections from 5.5-month-old 3xTg-AD mice intracranially injected with equivalent volumes of either T₁₂P₅(PS) NPs (2 mg/kg), PS-b-PEG(PS) control NPs, or saline. BG staining indicates preserved myelin sheath in T₁₂P₅(PS)-treated brains compared to control groups (saline and PS-b-PEG[PS]). Scale bar: 100 μm.

### DETAILED DESCRIPTION OF THE INVENTION

### 1. Overview

Unless otherwise defined, all technical and scientific terms used herein have the same meaning as commonly understood by one of ordinary skill in the art to which this invention pertains. In the case of conflict, the present document, including definitions will be controlled. This invention concerns nanoparticle compositions and treatment of Alzheimer's disease (early, mid, and late stage) and similar amyloidopathies using nanoparticles that target fibril β-amyloid (fAβ)-specific scavenger receptors. The invention further relates to using nanoparticles to inhibit fAβ-mediated pathology. In particular, the nanoparticles described herein can 1. block Aβ fibrilization; 2. promote Aβ clearance; 3. reduce internalization of Aβ fibrilization; and/or 4. suppress Aβ-induced microglial activation.

### 2. Nanoparticle Composition

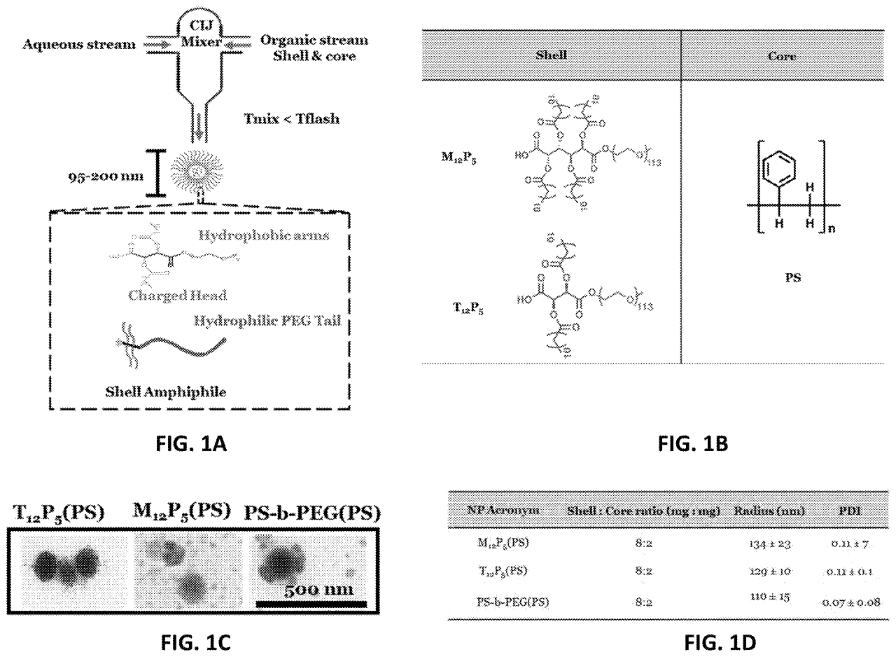

Provided are nanoparticle compositions that are amphiphilic macromolecules (AMs) composed of sugar-based backbones, aliphatic side chains, and hydrophilic poly(ethylene glycol) (PEG). Due to their amphiphilic nature, AMs can be complexed around hydrophobic core molecules via kinetic flash nanoprecipitation (FNP), forming nanoparticles (NPs) (FIG. 1A). NPs prepared by this technique display a greater resistance to particle dissolution and allow higher bioactive loading capacities in comparison to traditional micellar constructs.

The Flash NanoPrecipitation (FNP) process involves a confined impinging jet mixer (CIJ) to mix an aqueous stream with a water-miscible organic solvent stream containing hydrophobic solutes and amphiphilic macromolecules (AMs). Rapid mixing generates supersaturation of the hydrophobic solute in the anti-solvent stream, which induces nucleation and NP growth. Hydrophobic functionalities of the AM are also incorporated during growth. To ensure uniform kinetics and thus a homogenous NP size distribution, the time of mixing both streams, Tmix, must be less than the NP formation time, Tflash, or in other words the induction times of both AM aggregation and precipitation/nucleation of the core solute. In addition to having Tmix<Tflash, the solubilities of the AM hydrophobe and the core components must be properly matched, otherwise the rate of precipitation of either component will not proceed in tandem leading to colloidal instability and/or broad diameter distributions (See U.S. Pat. No. 10,016,517).

A bioactive nanoparticle produced by FNP comprises a hydrophobic core and bioactive amphiphilic macromolecules (bAMs). The bAM contains a hydrophilic and a hydrophobic segment. The hydrophobic segment of the bAM essentially coats the core of the NP, because the hydrophobic segment of the AM is physically embedded into the NP core by the physical means of Flash NanoPrecipitation. In one embodiment, the hydrophobic core and the bAM solubilities are properly matched. In other words, the hydrophobic segment of the bAM is properly matched to the hydrophobic core. In one embodiment, a NP comprises a hydrophobic core and a bioactive amphiphilic macromolecule coating said core and comprising a hydrophobic segment and a hydrophilic segment; wherein said nanoparticle comprises a single hydrophobic phase consisting of said hydrophobic core and said hydrophobic segment of the bioactive amphiphilic macromolecule; wherein the hydrophobic core and the bioactive amphiphilic macromolecule are selected so said hydrophobic core and the hydrophobic segment of said bioactive amphiphilic macromolecule are miscible in the hydrophobic phase.

Other information regarding the NPs can be found in U.S. Pat. Nos. 10,016,517, 9,434,681, the disclosures of each are incorporated by reference in their entirety.

In some embodiments, the NPs range in diameter from 90 nm to 200 nm such as 90 nm, 100 nm, 110 nm, 120 nm, 130 nm, 140 nm, 150 nm, 160 nm, 170 nm, 180 nm, 190 nm, 200 nm, or any value in-between. In one embodiment, the diameter of the NP is 120 to 140 nm such as 120 nm, 121 nm, 122 nm, 123 nm, 124 nm, 125 nm, 126 nm, 127 nm, 128 nm, 129 nm, 130 nm, 131 nm, 132 nm, 133 nm, 134 nm, 135 nm, 136 nm, 137 nm, 138 nm, 139 nm, 140 nm, or any value in-between. In one embodiment, the diameter of the NP is 90 nm to 160 nm such as 90 nm, 95 nm, 100 nm, 105 nm, 110 nm, 115 nm, 120 nm, 130 nm, 135 nm, 140 nm, 145 nm, 150 nm, 155 nm, 160 nm, or any value in-between. The diameter can be measured using any suitable analytical technique, such as, for example, dynamic light scattering.

In some embodiments, the NPs have a bioactive amphiphilic macromolecule that comprises a structure set forth in Formula A or Formula B:

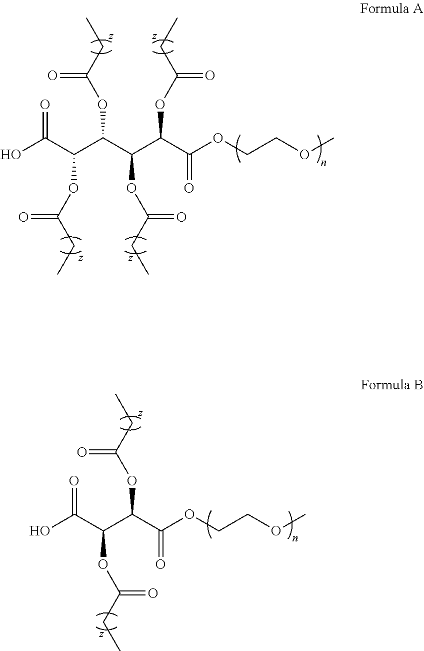

wherein n is an integer from 100 to 125; and
wherein z is an integer from 2 to 20. In some embodiments, wherein z is an integer from 4 to 12. In one embodiment, z is 10. In some embodiments, n is an integer from 110 to 120. In one embodiment, n is 113.

In some embodiments, the NPs have the structure depicted in FIG. 1B (i.e., the M₁₂P₅ or T₁₂P₅ shell and the PS core).

In some embodiments, the NPs have a hydrophobic core comprises a structure set forth in Formula C:

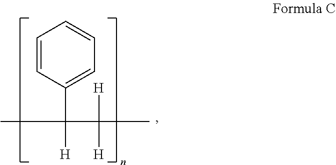

wherein n is an integer from 10 to 20. In some embodiments, m is an integer from 10 to 15.

In some embodiments, the NPs have a polydispersity index (PDI) of less than 0.3.

In some embodiments, the NPs bind to fibril β-amyloid (fAβ)-specific scavenger receptors. For example, the fAβ-specific scavenger receptors can be selected from the group consisting of CD36, CD68, and SRA1 (Steroid Receptor RNA Activator 1).

As used herein, a “hydrophobic core” is a hydrophobic molecule that is insoluble in aqueous solutions. The molecule may be without limitation a polymer, a carbohydrate, or a chain of peptides.

As used herein, the terms “polymer,” “polymeric” and similar terms have the usual meaning known to those skilled in the art and thus may be used to refer to homopolymers, copolymers (e.g., random copolymer, alternating copolymer, block copolymer, graft copolymer) and mixtures thereof.

Non-biodegradable or biodegradable polymers may be used. Representative polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups and other modifications routinely made by those skilled in the art.

As used herein, “a bioactive amphiphilic macromolecule” (bAM) is a hydrophilic-block-hydrophobic copolymer, for example, a hydrophobic carbohydrate-derived backbone modified with alkyl chains covalently bound to a hydrophilic polymer such as polyethylene glycol (PEG) (FIG. 1B).

As used herein, the term “phase” in regard to the NPs refers to a region of space, throughout which all physical properties of a material are essentially uniform. Phases may also be differentiated based on solubility as in polar (hydrophilic) or non-polar (hydrophobic). A mixture of water (a polar liquid) and oil (a non-polar liquid) will spontaneously separate into two phases. Here, the NP comprises a hydrophobic core and an AM that does not spontaneously separate into two phases and is characterized by a single hydrophobic phase. The AM is not bound chemically nor thermodynamically to the NP core. The AM can be frozen or locked to the NP core.

As used herein, the term “miscible” refers to the ability to be mixed into a uniform solution or phase.

As used herein and in the appended claims, the singular forms “a,” “and,” and “the” include plural references unless the context clearly dictates otherwise.

### 3. Nanoparticle Composition

The NP compositions described herein can be formulated as a pharmaceutical composition, and may be administered to a mammalian subject, such as a human.

As used herein, the term “subject” refers to an animal, preferably a mammal such as a human.

The pharmaceutical composition can be administered to a subject via various methods. For example, the pharmaceutical composition described herein can be administered intranasally, intraperitoneally, intravenously, intracranially, intracerebral, intraventricular, or through CSF infusion, or via lumbar CSF injection.

In particular, the NP compositions described herein exhibit inherent properties that do not impede their ability to cross the brain-blood barrier (BBB). In addition, NP compositions may be further modified to increase their ability to cross the BBB.

Any material used in preparing any unit dosage form should be pharmaceutically acceptable and substantially non-toxic in the amounts employed. To administer the pharmaceutical composition to a patient, it is preferable to formulate the molecules in a composition comprising one or more pharmaceutically acceptable carriers. As used herein the term “pharmaceutically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like. In addition, the compositions of the invention can be formulated into sustained-release preparations and devices.

The pharmaceutical dosage forms suitable for injection or infusion should be preferably sterile, fluid and stable under the conditions of manufacture and storage. The prevention of the action of microorganisms may be brought about by various antibacterial and antifungal agents, for example, parabens, chlorobutanol, phenol, sorbic acid, thimerosal, and the like. Others are also suitable. In many cases, it may be preferable to include isotonic agents, for example, sugars, buffers or sodium chloride.

Sterile injectable solutions may be prepared by incorporating the pharmaceutical composition of the invention in the required amount into an appropriate solvent or medium with various other ingredients, e.g., those enumerated above, as needed, which may be followed by sterilization.

In one embodiment, the pharmaceutical composition is delivered to a subject via a biomedical device. Accordingly, the biomedical device contains a pharmaceutical composition described herein comprising NPs.

As used herein the term “biomedical device” refers to an instrumentality to be implanted into or onto a subject in order to bring about a desired result. Examples of biomedical devices, include, but are not limited to, stents, grafts, shunts, stent grafts, fistulas, angioplasty devices, balloon catheters, venous catheters, implantable drug delivery devices, adhesion barriers, hydrogels, biological polymers, microelectrodes, probes, prosthetic valve, a cardiac valve, or a venous valve and tissue scaffolds.

### 2. Method of Treatment

In one embodiment, provided is a method for inhibiting amyloid beta (Aβ) fibrilization in a subject in need thereof (e.g., a human), comprising administering a therapeutically effective amount of a NP, wherein the bioactive amphiphilic macromolecule of the NP comprises a structure set forth in Formula A or Formula B as described herein (FIG. 1B), or a pharmaceutically acceptable salt thereof, to the mammal. In one embodiment, the bioactive amphiphilic macromolecule of the NP comprises a structure set forth in Formula A. In one embodiment, the bioactive amphiphilic macromolecule of the NP comprises a structure set forth in Formula B.

In one embodiment, provided is a method of treating Alzheimer's disease (AD) and similar amyloidopathies in a subject in need thereof, comprising administering a therapeutically effective amount of a NP, wherein the bioactive amphiphilic macromolecule of the NP comprises a structure set forth in Formula A or Formula B as described herein (FIG. 1B), or a pharmaceutically acceptable salt thereof, to the mammal. In one embodiment, the bioactive amphiphilic macromolecule of the NP comprises a structure set forth in Formula A. In one embodiment, the bioactive amphiphilic macromolecule of the NP comprises a structure set forth in Formula B. In some embodiments, a NP described herein is used to treat Alzheimer's disease. In some embodiments, the Alzheimer's disease is early-stage Alzheimer's disease, moderate Alzheimer's disease, or late-stage Alzheimer's disease. Similar amyloidopathies to Alzheimer's disease include, but are not limited to hereditary cerebral amyloid angiopathy (such as familial British dementia and familial Danish dementia).

In some embodiments, provided is a method of treating neuroinflammation-associated brain diseases comprising administering a therapeutically effective amount of a NP, wherein the bioactive amphiphilic macromolecule of the NP comprises a structure set forth in Formula A or Formula B as described herein (FIG. 1B), or a pharmaceutically acceptable salt thereof, to the mammal. In one embodiment, the bioactive amphiphilic macromolecule of the NP comprises a structure set forth in Formula A. In one embodiment, the bioactive amphiphilic macromolecule of the NP comprises a structure set forth in Formula B. Examples of treating neuroinflammation-associated brain diseases include, but are not limited to stroke, traumatic brain injury, and amyotrophic lateral sclerosis (ALS).

As used herein the terms “treating” or “treatment” refers to administration of the nanoparticles or pharmaceutical composition to a subject or patient, who has Alzheimer's disease (early, mid, or late-stage), with the purpose to cure, alleviate, relieve, remedy, delay the onset of, or ameliorate the disorder, the symptom of the disorder, the disease state secondary to the disorder, or the predisposition toward the disorder.

As used herein the term a “therapeutically effective amount” refers to the amount of the nanoparticles or pharmaceutical composition sufficient to effect beneficial or desired results. A therapeutically effective amount can be administered in one or more administrations, applications or dosages and is not intended to be limited to a particular formulation or administration route.

An NP pharmaceutical composition described herein can be administered in vivo or ex vivo, alone or co-administered in conjunction with other therapeutic agents. As used herein, the term “co-administration” or “co-administered” refers to the administration of at least two therapeutic agents to a subject. In some embodiments, the co-administration of two or more therapeutic agents is concurrent. In other embodiments, a first therapeutic agent is administered prior to a second therapeutic agent. Those of skill in the art understand that the formulations and/or routes of administration of the various therapeutic agents used may vary.

In some embodiments, the subject has previously received treatment for Alzheimer's disease or amyloid beta (Aβ) fibrilization with an existing therapeutic agent.

Examples of existing therapeutic agents include, but are not limited to lecanemab (LEQEMBI®), aducanumab (ADUHELM®), memantine (Ebixa, Axura, NAMENDA®, Akatinol), cholinesterase inhibitors (such as donepezil, rivastigmine, and galantamine), memantine HCl and donepezil HCl (NAMZARIC®), and brexpiprazole (REXULTI®).

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100 mg/kg. Variations in the needed dosage are to be expected in view of the variety of compounds available and the different efficiencies of various routes of administration. Variations in these dosage levels can be adjusted using standard empirical routines for optimization as is well understood in the art.

In some embodiments, an NP pharmaceutical composition is administered at a dose of about 0.1 mg/kg, about 1.0 mg/kg, about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 500 mg/kg of body weight, or any dose in-between.

In some embodiments, an NP pharmaceutical composition is administered at a dose of about 10 μg to about 1 gram. For example, the NP pharmaceutical composition can be administered to a subject in need thereof in amount of about 10 μg, about 20 μg, about 30 μg, about 40 μg, about 50 μg, about 60 μg, about 70 μg, about 80 μg, about 90 μg, about 100 μg, about 200 μg, about 300 μg, about 400 μg, about 500 μg, about 600 μg, about 700 μg, about 800 μg, about 900 μg, about 1000 μg, about 100 mg, about 200 mg, about 300 mg, about 400 mg, about 500 mg, about 600 mg, about 700 mg, about 800 mg, about 800 mg, about 900 mg, about 1 gram (1000 mg), or any amount in-between.

It will be appreciated by those skilled in the art that changes could be made to the embodiments described above without departing from the broad inventive concept thereof. It is understood, therefore, that this invention is not limited to the particular embodiments or examples disclosed, but it is intended to cover modifications that are within the spirit and scope of the present invention as defined by the appended claims.

### EXAMPLES

### Example 1. Materials and Methods

This Examples details the materials and methods used in Examples 2 to 5.

### Example 1A. Synthesis of Amphiphilic Macromolecules

Macromolecular shells M₁₂P₅ and T₁₂P₅ were synthesized as previously described via esterification reactions conducted in two steps (Zhao, N et al. Advanced Nanobiomed Research. 2022;2(6):2100120; Djordjevid, J P et al. Journal of Bioactive and Compatible Polymers. 2008; 23:532-51; and Iverson, N M et al. Acta Biomaterialia. 2010; 6(8):3081-91). Briefly, the first step of M₁₂P₅, synthesis was performed by reacting mucic acid (20 mmol), zinc chloride (2 mmol) and lauroyl chloride (160 mmol) at 90° C. under inert atmosphere for 12 hours. Diethyl ether (20 mL) was added to the reaction mixture after it was cooled to room temperature. The mixture was then poured over ice-cold water (150 mL) while stirring. Then 80 mL of diethyl ether was added to the mixture and stirred continuously for 30 min. Extractions with brine were performed until the aqueous layer reached pH ~7. The organic layer was separated, dried over sodium sulfate, and evaporated under reduced pressure. Purification was performed by dissolving the crude product in diethyl ether (20 mL) and precipitation into petroleum ether (200 mL). Pure product (M₁₂) was isolated by vacuum filtration. For the first step of T₁₂P₅ generation, tartaric acid (7 mmol) and zinc chloride (2.2 mmol) were suspended in lauroyl chloride (52.5 mmol) and allowed to stir for 24 hours under inert atmosphere at 95° C. Then, DI (deionized)-water (30 mL) and diethyl ether (100 mL) were added to quench the reaction mixture and stirring continued for 30 min at room temperature. Five extractions were performed using DI-water (100 mL/wash). The organic layer was separated, dried over magnesium sulfate, and concentrated under reduced pressure. Brown liquid obtained was precipitated over hexanes and pure product (T₁₂) was isolated by vacuum filtration. The second step of the synthesis consisted in the PEGylation reaction of the M₁₂ and T₁₂ via carbodiimide chemistry to generate M₁₂P₅ and T₁₂P₅, consecutively. For the PEGylation reaction, M₁₂ or T₁₂ (0.45 mmol) and DPTS (4-(dimethylamino)pyridinium 4-toluenesulfonate, 0.15 mmol) were dissolved in anhydrous dichloromethane (DCM, 10 mL) and anhydrous dimethylformamide (DMF, 3 mL) under inert atmosphere at room temperature. mPEG(5k) was added to the reaction mixture and after complete dissolution, N,N-diisopropylcarbodiimide (DIC, 0.48 mmol) was added drop-wise and stirred continuously for 48 hours under argon. Next, the reaction mixture was cooled down at −20° C. and the white solid (side product) was precipitated and removed by vacuum filtration. Extra DCM (25 mL) was added to the filtrate and extractions with hydrochloric acid HCl (0.1M, lx 40 mL) and brine (2×40 mL) were performed as part of the purification. After separation from the aqueous layer, the organic layer was dried over magnesium sulfate and solvent was evaporated under reduced pressure. The product was dissolved in diethyl ether (50 mL) and isolated by centrifugation (1370×g, 5 min). Products were dried under vacuum and characterized using 1H NMR-spectroscopy, Fourier Transform Infrared (FTIR) spectroscopy and differential scanning calorimetry.

### Example 1B. Nanoparticle fabrication and characterization

NPs were synthesized and characterized as previously described using established techniques (Zhao, N et al. Advanced Nanobiomed Research. 2022;2(6):2100120 and Djordjevid, J P et al. Journal of Bioactive and Compatible Polymers. 2008; 23:532-51). NPs were fabricated using a flash nanoprecipitation (FNP) technique (FIG. 1A) as previously reported (Zhao, N et al. Advanced Nanobiomed Research. 2022;2(6):2100120; York, A W et al. Adv Mater. 2012; 24(6):733-9; Lewis, D R et al. PNAS. 2015; 112(9):2693-8). In brief, a confined impinging jet mixer was used to mix a stream of 250 mL of 50/50% (v/v) mixture of tetrahydrofuran (THF) (Sigma) containing 8 mg/ml shell molecule, and 2.5 mg/ml hydrophobic core molecule with 250 mL of an aqueous stream. To ensure homogenous NPs size distribution, the time of NPs formation (Tflash) was prescribed to be more than the time of mixing both streams (Tmix). For fluorescent NPs, 3.75% 1,1 0-dioctadecyl-3,3,3 0,3 0,3 0-tetramethylindocarbocyanine perchlorate (Dil) (Thermo Fisher Scientific) was mixed with the organic stream of shell and core mixture. The exit stream was immediately introduced into a 9-fold volume of water, then NPs were dialyzed against water using 3.5 KDa MW cutoff dialysis cassette (Thermo Fisher). NPs hydrodynamic radius, polydispersity index (PDI) were characterized using dynamic light scattering (DLS) (a Malvern-Zetasizer Nano ZS90 series DLS detector) (York, A W et al. Adv Mater. 2012; 24(6):733-9). The critical micelle concentration, size, and charge data has been published in the literature (Zhao, N et al. Advanced Nanobiomed Research. 2022;2(6):2100120; York, A W et al. Adv Mater. 2012; 24(6):733-9; and Wang, J et al. Int. J. Nanomedicine. 2007; 2(4):697-705). Example 1C.

Preparation and characterization of Aβ 1-42 fibrils

Aβ1-42 fibrils were generated as previously reported with slight modifications (Ryan, D A et al. J Neurosci Methods. 2010; 190(2):171-9 and Sondag, C M et al. Journal of Neuroinflammation. 2009;6(1):1). Briefly, human Aβ₁₋₄₂ (Anaspec) was suspended in 100% 1,1,1,3,3,3-hexafluoro-2-propanol to a final concentration of 5 mg/mL, aliquoted and then dried at room temperature overnight in a fume hood. The aliquoted peptide was dissolved in DMSO to a final concentration of 5 mM and sonicated for 1 min in low binding microcentrifuge tubes (Corning). The Aβ₁₋₄₂ peptide was diluted in phosphate-buffered saline (PBS) and 0.2% sodium dodecyl sulfate to 200 μM; next, fibrils were prepared by incubating the diluted peptide for at least 4 weeks at 37° C. with constant shaking at 300 rpm. Fibrils were separated after centrifugation for 1 hour at 5000× g at 4° C. The concentration of Aβ fibrils was determined by measuring absorbance at 280 nm and calculated using extinction coefficient at 1280M−1 cm−1 (Xue, C et al. R Soc Open Sci. 2017;4(8):170325). Fibril formation was verified using thioflavin-T (Th-T) fluorescence assay and transmission electron microscopy (TEM).

### Example 1D. Thioflavin-T (Th-T) Fluorescence Assay

Amyloid beta fibril formation was assessed using Th-T fluorescence assay (Xue, C, et al. Roylal Society Open Science. 2017;4(1):160696). First, 3 mM stock of Th-T (Sigma) was prepared and filtered through 0.2 μm syringe filter. To measure Aβ fibril formation, 40 μM Th-T was mixed with 5 μL of either PBS (control) or the protein. Using black bottom 96 well microplate (Corning), the fluorescence intensity was measured at room temperature using Tecan Infinite M200 Pro microplate reader at excitation 450 nm and emission 485 nm. Results were presented as a ratio of the control (Th-T plus PBS) reading.

### Example 1E. Transmission Electron Microscopy

To prepare fAβ sample for TEM imaging, a negative staining protocol was conducted as described before. Briefly, 5 μL of 20 M fAβ was loaded on a formvar-coated, carbon-stabilized copper grid (400 mesh, Pacific Grid-tech). Excess solution was drained using a Whatman filter paper. The grid was washed and negatively stained with 5 μL of 2% uranyl acetate. Excess solution was drained, and the grid imaged using Philips CM12 electron microscope with AMT-XR11 digital camera. The images were acquired at magnifications of 60,000× at 80 kV. For NPs imaging, NPs were loaded on a formvar-coated, carbon-stabilized copper grid (400 mesh, Pacific Grid-tech). Excess solution was drained using a Whatman filter paper then NPs were directly imaged.

### Example 1F. Kinetics of Aβ Fibrillization

The effect of AM-NPs on Aβ fibrilization was assessed as previously described (Xue, C et al. R Soc Open Sci. 2017;4(8):170325). Samples containing either 8 μM monomeric Aβ only or in presence of NPs at 1:10 volume ration were loaded with 20 μM Th-T into 96 well clear bottomed non-binding half-area plates (Corning). Samples of NPs only plus Th-T were measured to account for the background reading due to NPs. Plates were sealed with Axygen sealing tape (Corning) and the fluorescence intensity was monitored over 63 h using Tecan Infinite® M200 PRO microplate reader at excitation 450 nm and emission 485 nm while agitated at 600 rpm at 37° C. The reading of each sample containing Aβ plus NPs was subtracted from the fluorescence of the corresponding sample of each NP then normalized to the Aβ plus Th-T reading.

### Example 1G. Cell Culture

BV2 mouse microglia cell line: BV2 microglia were kindly provided by Drs. Bin Liu (University of Florida) and Jason Richardson (Northeast Ohio Medical University). BV2 microglia were cultured in Dulbecco's Modified Eagle Medium (DMEM) (Gibco) supplemented with 10% fetal bovine serum (FBS) and 1% penicillin-streptomycin (Pen/Strep) (Gibco). Cells were plated in 96 well plate at 20,000/well and allowed to adhere to plates for 24 h before any treatment. All treatments were conducted in 1% FBS and 1% Pen/Strep DMEM media.

SH-SY5Y human neuroblastoma cell line: SH-SY5Y (ATCC) cells were plated in 96 well plate at 15,000 cells/per well and allowed to adhere overnight in DMEM supplemented with 10% FBS and 1% Pen/Strep. All treatments were conducted in 1% FBS and 1% Pen/Strep DMEM media.

### Example 1H. Cell-based Competitive Receptor Binding Assay

To screen for SRs that competitively bind to the AM-NPs described herein, the efficacy of AM-NPs to compete with CD36, CD68, SRA1, and TLR2 specific antibodies on BV2 cell surface was tested. BV2 microglia were plated at 20,000 cells per well in a 96-well plate for 24 h. Using 0.2% sodium azide in DMEM, cells were incubated with 1:50 diluted NPs for 1 h, then co-incubated with SR-specific antibody or isotype controls for 30 min. Cells were fixed with 4% paraformaldehyde (PFA) (Sigma) for 15 min, then washed two times with PBS. Cells were then blocked with 2% goat serum without triton-X-100 to avoid permeabilization of the plasma membrane. To avoid internalization of NPs or antibodies, cells were incubated on ice and in the presence of sodium azide until imaged. Cells were incubated with secondary antibodies including Alexa 488 or 594 (Life Technologies) for 1 h at room temperature. Cells were washed with PBS then counterstained with Hoechst (Thermo Fischer) to visualize nuclei. Cells untreated with NPs were used as a control for each SR. Primary antibodies were CD36 (Abcam), CD68 (Biolegend), SRA1 (Proteintech), and TLR2 (Novus). Cells were imaged on a Zeiss LSM 780 confocal microscope using a 20× objective. Extracellular fluorescence quantification was performed using FIJI/ImageJ software by measuring mean grey value in cells segmented by applying the same fluorescence thresholds to all collected images. Fluorescence of each field was divided by the number of cells in the field then normalized to the control.

### Example 11. Aβ Internalization Assay

To screen for SRs that mediate fAβ internalization and to determine if AM-NPs modulate fAβ internalization in BV2 cells, microglia were plated at 20,000 cells per well in a 96-well plate. BV2 microglia were co-incubated with 20 M fAβ mixed with 0.035 wt % HiLyte™ Fluor 488 labeled fAβ (for imaging purposes) for 24 h after pre-incubation in the presence or absence of SR-specific antibodies, isotype controls, or AM-NPs for 24 h. Cells were fixed with 4% PFA then washed two times with PBS to remove extracellular fAβ. Cells were then incubated with 0.5% triton-X-100 (Sigma) in potassium buffered saline (PBS-T) to remove membrane-bound fAβ particles. Primary antibodies were CD36 (Abcam), CD68 (Biolegend), SRA1 (Proteintech), and TLR2 (Novus). Cells were imaged on a Zeiss LSM 780 confocal microscope using a 20× objective. Intracellular fluorescence quantification was performed using FIJI/ImageJ software by measuring mean grey value in cells segmented by applying the same fluorescence thresholds to all collected images. For untreated cell controls, fluorescence images were segmented based on cell boundaries visualized in brightfield images.

### Example 1J. TNF-α Cytokine and Nitric Oxide Assay

BV2 microglia were plated at 20,000 cells per well in a 96 well plate and allowed to adhere overnight. Cells were co-treated with 20 μM fAβ in the presence or absence of NPs or 10 ng/ml lipopolysaccharide (LPS). After 24 h, the supernatants were collected and assayed for TNF-α production using an ELISA assay (R&D systems). Nitric oxide production in the supernatants was measured using Griess reagent (Promega). Cells were fixed using 4% PFA then washed with PBS then used to assay the intracellular iNOS as described in immunocytochemistry (ICC) below.

### Example 1K. Immunocytochemistry

Cells were fixed with 4% PFA for 15 min, then washed with PBS. Cells were permeabilized in PBS-T for 10 min, then blocked for 1 h with 2% goat serum (MP Biomedicals) blocking buffer at room temperature. Cells were incubated with primary antibodies including iNOS (Abcam), and NF-κB (Santa Cruz) overnight at 4° C., then washed with PBS-T. Cells were incubated with secondary antibodies including Alexa 488, or 594 (Life Technologies) for 1 h at room temperature. Cells were washed with PBS then counter stained with Hoechst (Thermo Fischer) to visualize nuclei. For imaging extracellular or cell surface proteins, Triton-X-100 was eliminated from the protocol. Cells were imaged using Zeiss LSM 780 confocal microscope using 20× and 40× water immersion objectives.

### Example 1L. Lysosome Activity

To study the activity of the acidic lysosomes, BV2 microglia were pre-treated with or without NPs for 24 h. Then cells were co-treated with fAβ488 for either 2 h or 24 h. Cells were then washed and incubated in 70 μM LysoTracker™ Red DND-99 (ThermoFisher) for 30 min prior to fixation with 4% PFA. Images were captured at multiple focal planes via Zeiss LSM 780 confocal microscope using 20× and 40× objectives. The colocalization of fAβ3488 with lysosomes was performed using Mander's overlap coefficient as previously described (Bolte, S et al. J Microsc. 2006; 224(3):213-32). Immunostaining for CD68 (Biolegend) and the lysosomal associated protein-1 (LAMP-1) (Invitrogen) was performed (as described above) in BV2 microglia co-treated with 20 μM fAB488 and Dil-labeled NPs for 2 h after treatment with or without NPs for 24 h.

### Example 1M. Autophagic Activity

To study the autophagic activity, BV2 microglia were pre-treated with or without NPs for 24 h. Then cells were co-treated with fAβ for 30 min prior to fixation with 4% PFA for ICC. For TEM imaging, cells were fixed with a mixture of 2.5% Glutaraldehyde, 4% PFA in 0.1 M Cacodylate buffer at pH 7.4. Cells were stained against the autophagosome marker LC3B (Invitrogen) and nuclei were counterstained using Hoechst according to the ICC protocol described above.

### Example 1N. NF-κB Translocation Assay

To investigate the nuclear translocation of NF-κB in BV2 cells, an immunostaining assay was conducted as previously described using confocal microscopy (Trask, OJ, Jr. Nuclear Factor Kappa B (NF-κB) Translocation Assay Development and Validation for High Content Screening. In: Markossian, S et al., editors. Assay Guidance Manual. Bethesda (MD): Eli Lilly & Company and the National Center for Advancing Translational Sciences; 2004). 20 μM fAβ-treated BV2 cells were co-incubated with or without AM-NPs for 2 h then immediately fixed with 4% PFA. Using the immunocytochemistry assay described above, the focal location of the cell nucleus was determined with Hoechst stain and used to define the nuclear regions of interest (ROI). The mean nuclear NF-κB fluorescence intensity was measured in ROI while the cytoplasmic NF-κB fluorescence intensity was measured after subtracting ROI from the imaging field. Then the ratio of nuclear to cytoplasmic NF-κB fluorescence was calculated.

### Example 10. Image Acquisition

Images of BV2 cells were obtained with a Carl Zeiss LSM 780 confocal microscope. Lasers for image acquisition were Argon Ion for Alexa Fluor 488 nm probe, HeNe for Alexa Fluor 647 nm probe configuration. Complementary DIC (differential interference contrast was used for focusing, imaging, and analysis purposes. For each configuration, detectors were adjusted to eliminate spectral overlap between channels to unique bandwidth. To further minimize the spectral bleed-through, images were taken for each fluorescent probe in sequential mode.

### Example 1P. Neurotoxicity Assay

BV2 microglia were plated in 96 well plate at 20,000 cells per well, and SH-SY5Y were plated separately in 96 well plate at 15,000 cells per well. Microglia were treated with 20 μM fAβ in the presence or absence of NPs for 24 h. In parallel, other wells were either treated with vehicle (1% FBS media plus PBS) or with 10 ng/ml LPS. The BV2-conditioned media (CM) from this experiment was harvested and used to treat SH-SY5Y cells for 24 h. The neurotoxicity in response to BV2-CM was quantified in SH-SY5Y cells using lactate dehydrogenase (LDH) (Promega), normalized to fAB-treated SH-SY5Y cells, and to untreated BV2-CM SH-SY5Y cells.

### Example 10. Statistical Analysis

Data were presented as mean±SEM unless otherwise indicated, from at least 3 independent experiments (n>3). Analysis was performed using student's t-test, One-Way Analysis Of Variance (One-Way ANOVA), or Two-Way ANOVA followed by pairwise multiple comparisons test, where p<0.05 was statistically significant.

### Example 2. Investigation of AM-NPs' ability to prevent amyloid beta fibrilization

Three sets of AM-NPs at equivalent concentrations with identical polystyrene (PS) core and comparable shell molecular mass concentrations were fabricated (FIGS. 1A and 1B). The first set (T₁₂P₅(PS)) is composed of a bioactive tartaric acid-derived shell (T₁₂P₅) and a non-bioactive core of PS. The second set (M₁₂P₅(PS)) is composed of a bioactive mucic acid-derived shell (M₁₂P₅), and a non-bioactive core of PS. The third set (PS-b-PEG(PS)) are equivalently sized control NPs composed of comparable molecular weight non-bioactive shell of polystyrene-block-poly(ethylene glycol) (PS-b-PEG) and PS core (FIGS. 1A and 1B). NPs were fabricated via FNP (FIG. 1A), where fast mixing speed allows the formation of nano-assemblies. Using different ratios of shell-to-core weight, the optimal ratio that ensures the formation of non-aggregating and stable particles were used (FIG. 1C). These results indicated that using the shell-to-core ratio of 8:2, resulted in the generation of stable NPs with radii ranging from 90 to 200 nm (FIG. 1C). These NPs were characterized by a low polydispersity index (PDI), less than 0.3, indicating relatively monodisperse distribution in an aqueous solution (FIG. 1C).

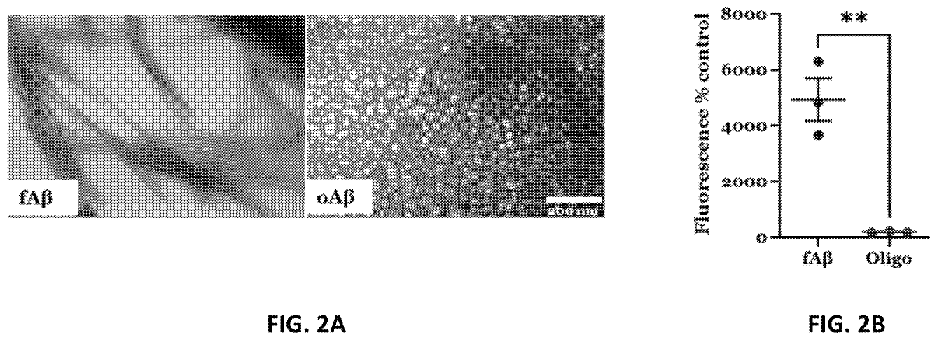

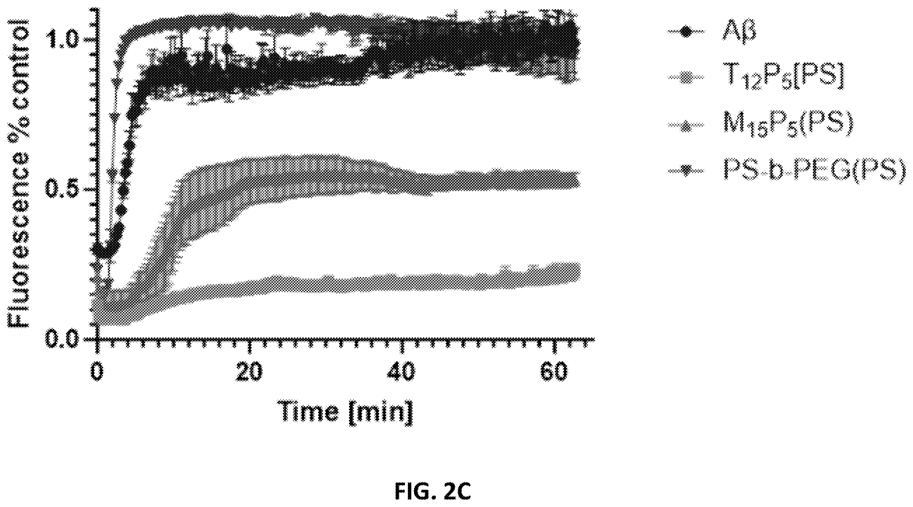

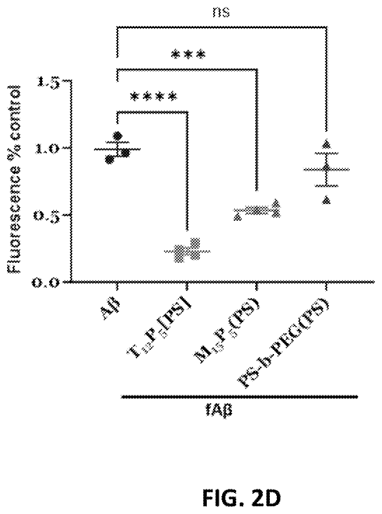

Next, fAβ from human Aβ1-42 peptide was synthesized, which is the most abundant and toxic form of Aβ in AD patients (Glenner, G G et al. Biochemical and Biophysical Research Communications. 1984; 120(3):885-90), in vitro using an established protocol with some modifications (Stine, W B et al. Methods Mol Biol. 2011; 670:13-32). The ultrastructure of the produced fAβ was verified and differentiated from the soluble oligomeric Aβ (oAβ) using TEM (FIG. 2A), and the degree of fibrilization of fAβ was assessed using a Th-T assay (FIG. 2B). To examine whether AM-NPs can interrupt the fibrilization of Aβ peptide, soluble Aβ peptide was incubated with Th-T in the presence or absence of NPs; T₁₂P₅ (PS), M₁₂P₅(PS), or control PS-b-PEG(PS) at 37° C. Aromatic compounds such as thioflavin T/S and Congo red can selectively bind to β-sheet-rich fibrils; therefore, they have been widely used probes to detect Aβ fibrils in AD brain tissues, in vivo, and in vitro studies (LeVine H. Methods in Enzymology: Academic Press; 1999. p. 274-84 and Urbanc, B et al. PNAS. 2002; 99(22):13990-5). The binding of Th-T to fibrillar β sheets of fAβ stops the bond rotation of Th-T, therefore, recovers its emission at 485 nm once excited (Naiki, H et al. Analytical Biochemistry. 1989; 177(2):244-9). These results indicated the fibrilization kinetics of Aβ in Th-T (FIG. 2C), which is characterized by the commonly observed sigmoidal growth curve of Aβ fibrilization, which composed of three phases: lag phase, growth phase, and an equilibrium phase (Evans, K C et al. PNAS. 1995; 92(3):763-7). Interestingly, AM-NPs with bioactive shells T₁₂P₅(PS) and M₁₂P₅(PS), significantly shortened the growth phase of Aβ fibrilization and slowed the aggregation kinetics (FIG. 2C). In contrast, the control NPs PS-b-PSG(PS) did not impact the kinetics of Aβ aggregation (FIG. 2C). Also, the endpoint fluorescence of Th-T at 65 h was significantly reduced by 76% for T₁₂P₅(PS) (p<0.0001) and 47% for M₁₂P₅(PS) (p=0.0011) compared to control fAβ samples in Th-T (FIG. 2D). These results suggest that the bioactive shells on AM-NPs are sufficient to slow the transition from Aβ monomers to mature fibrils. Moreover, because AM-NPs have significantly reduced the endpoint fluorescence of Th-T, which is directly proportional to the fibril content (Naiki, H et al. Analytical Biochemistry. 1989; 177(2):244-9), these data indicate that the bioactive shells are able to reduce the amount of fAβ formed.

The above-presented results demonstrate that the sugar-based anionic AM-NPs can slow the transition from Aβ monomers to mature fibril and reduce the amount of fAβ formed under conditions that favor fibrilization (FIGS. 2A-2D). This anti-amyloidosis effect of NPs may be mediated by the electrostatic and hydrophobic interactions between the anionic aliphatic AM shells and the cationic lysine residues in Aβ monomers, which have been shown to play important role in the assembly and toxicity of Aβ and alpha-synuclein (ASYN) (Marshall, K E et al. Biochemistry. 2011; 50(12):2061-71 and Marshall, K E et al.

Prion 2014; 8(2):192-6). Both AMs M₁₂P₅ and T₁₂P₅ are lipophilic anionic molecules composed of mucic acid and tartaric acid backbones with aliphatic side chains and acidic end groups. This interaction may lower the concentration of the free monomers and shift the equilibrium away from fibrilization.

Another possibility is that the interactions between AM-NPs and oAβ may be strong enough to interrupt monomers binding, slowing the nucleation and fibril formation. Under physiological pH, Aβ exhibits net negative charges; however, the positively charged moieties in Aβ play a critical role in seeding and self-assembly (Marshall, K E et al. Biochemistry. 2011; 50(12):2061-71). In line with these findings, it was shown that the anionic lysine-specific molecular tweezers efficiently inhibit fibrilization of proteins, including Aβ, tau, and ASYN, through hydrophobic and electrostatic interactions (Sinha, S et al. J Am Chem Soc. 2011; 133(42):16958-69 and Acharya, S et al. J Biol Chem. 2014; 289(15):10727-37). Furthermore, targeting His13-Lys16 cluster region of Aβ using inorganic anionic compounds was found to inhibit Aβ aggregation through electrostatic and hydrophobic interactions (Geng, J et al. Angew Chem Int Ed Engl. 2011; 50(18):4184-8). Using polyphenols, hydrophobic interactions with the hydrophobic core in Aβ were sufficient to inhibit protein aggregation, promote disaggregation of preformed Aβ fibrils, and reduce the associated cytotoxicity (Olajide, O A et al. Inflammopharmacology 2020; 28(6):1439-55). Consistent with this notion, tannic acid was found to inhibit ASYN aggregation through hydrophobic interactions (Zhao, N et al. Frontiers in Bioengineering and Biotechnology. 2020; 8:112).

### Example 3. Evaluation of the Ability of AM-NPs to Bind to fAβ-Binding Scavenger Receptors And Interrupt fAβ Internalization

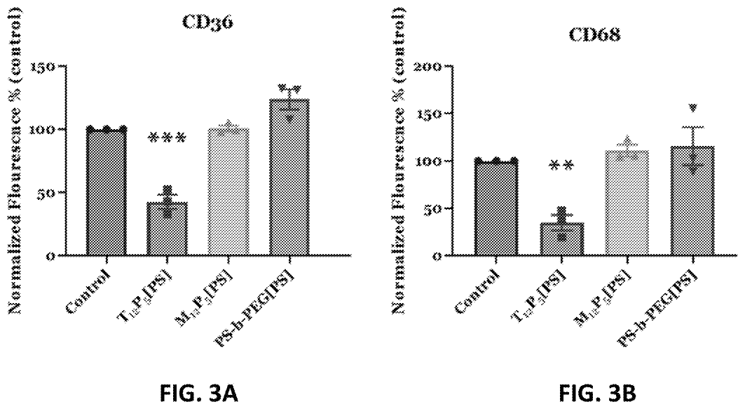

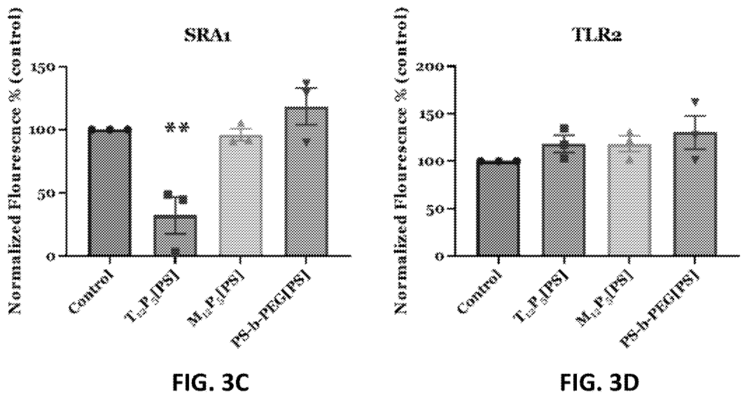

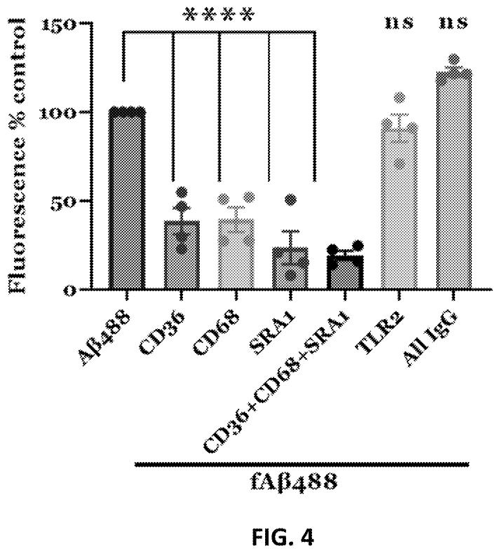

To investigate the role of AM-NPs in modulating fAβ trafficking in microglia through SRs, NP specificity to fAβ-specific SRs was screened using an immortalized BV2 murine cell line as a cell model. BV2 cells have been widely used over the past three decades to recapitulate the inflammatory response associated with neurodegenerative disease as observed in primary microglia (Henn, A et al. Altex. 2009; 26(2):83-94). To date, a wide range of SRs have been identified to play a role in fAβ pathology, of these receptors; CD36, CD68, SRA1, and TLRs. Previous molecular modeling and docking approaches confirmed specificity of AM shells T₁₂P₅ and M₁₂P₅ to SRs such as CD36 and SRA1 (Zhao, N et al. Advanced Nanobiomed Research. 2022;2(6):2100120 and Plourde, N M et al. Biomacromolecules. 2009; 10(6):1381-91). Using a cell-based competitive receptor binding assay, it was observed that NPs with tartaric acid-derived shell (T₁₂P₅(PS)) compete with SR-specific antibodies of CD36, CD68, and SRA1 and significantly reduced the surface fluorescence of these receptor, (p=0.0001), (p=0.009), and (p=0.0048), respectively (FIGS. 3A-C). However, these NPs did not exhibit receptor-binding activity to TLR2 in this cell line. On the other hand, NPs with mucic acid-derived NPs (M₁₂P₅(PS)) and control NPs (PS-b-PEG(PS)) did not exhibit appreciable specificity to any of the SRs on microglia screened in this cell-based assay. Next, the possible role of the SRs screened above was investigated in mediating the internalization of fAβ into microglia. Cells were co-incubated with fAβ and Alexa-labeled fAβ488 in the presence or absence of receptor-specific full-length antibodies or their isotype controls at 37° C. for 24 h (FIG. 4). Cells were fixed with 4% PFA then washed two times with PBS to remove extracellular fAβ. Cells were then incubated with 0.5% triton-X-100 in potassium buffered saline (PBS-T) to remove any membrane-bound fAβ particles.

The fluorescence of fAβ488 was quantified and compared to the control cells treated only with fAβ488 (FIG. 4). Cells blocked with CD36, CD68, and SRA1 receptor-specific antibodies were the least to internalize fAβ3 38%, 39%, and 23%, respectively (****p<0.0001) (FIG. 4). This observation was confirmed in cells treated with a mixture of the three receptor-specific antibodies of CD36, CD68, and SRA1, where the three antibodies exert an additional inhibitory effect on fAβ internalization (****p<0.0001). These data indicated that SRs, including CD36, CD68, and SRA1, but not TLR2, are essential for the internalization of fAβ. Therefore, targeting these SRs, singly or in combination, can be a potential approach to interrupt fAβ-mediated pathology.

Thus, the above-discussed results provide proof-of-concept about the involvement of CD68 in the trafficking of fAβ into microglia. CD68 is the only known member of the class D scavenger receptors that has been widely exploited as a macrophage marker. It belongs to the lysosomal associated-membrane proteins (LAMP) located mainly in the lysosomal membrane, but can rapidly shuttle to the cell surface where it binds to the oxidized low density lipoproteins, apoptotic cells, and phosphatidylserine (Chistiakov, D A et al. Laboratory Investigation. 2017; 97(1):4-13; Ramprasad, M P et al. PNAS. 1996; 93(25):14833-8; and Kurushima, H et al. J Leukoc Biol. 2000; 67(1):104-8). The tartaric acid-derived AM-NPs were shown to competitively bind to SRA1, CD36, and CD68 in the presence of receptor-targeting antibodies. Thus, T₁₂P₅(PS) can efficiently block CD68 and inhibit the binding with anti-CD68 antibody by 75%.

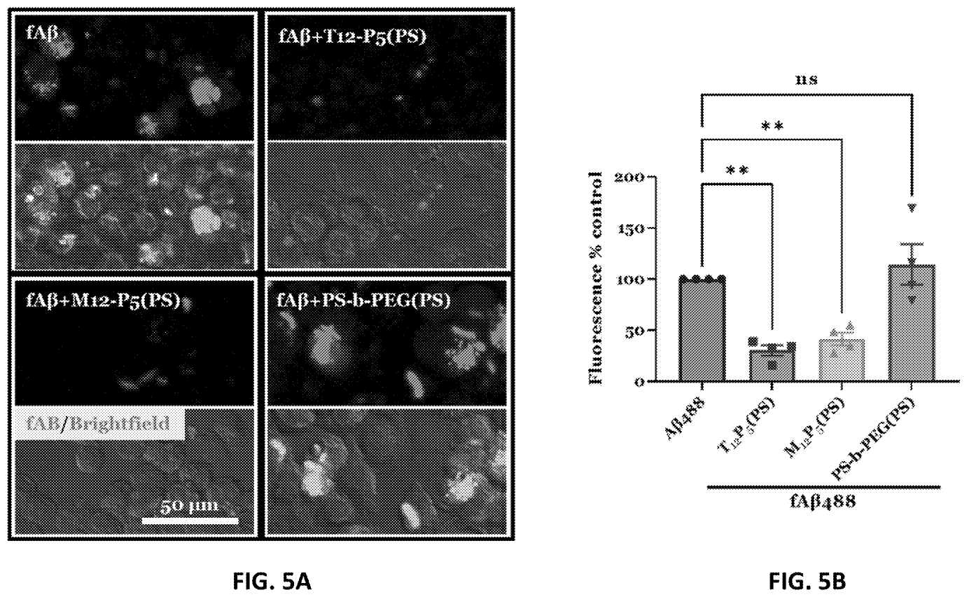

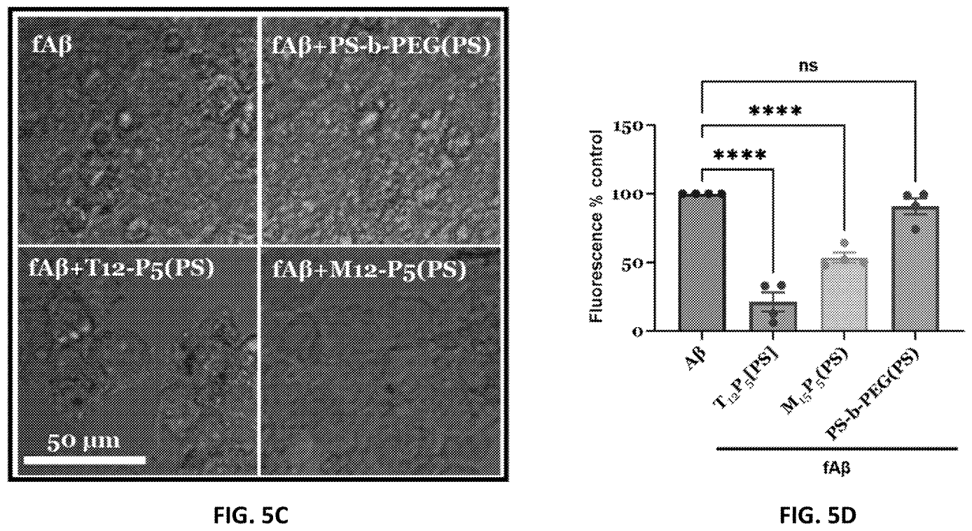

Next, given the molecular modeling and the in vitro results shown earlier, it was determined whether NPs can interrupt the microglial uptake of fAβ. Cells were co-incubated with fAβ and Alexa-labeled fAβ3488 in the presence or absence of AM-NPs or their NP control (PS-b-PEG(PS)) at 37° C. for 24 h (FIGS. 5A-5B). Cells were fixed with 4% PFA then washed two times with PBS to remove extracellular fAβ. Cells were then incubated with 0.5% triton-X-100 in potassium buffered saline (PBS-T) to remove any membrane-bound fAβ particles.

The fluorescence of fAβ488 was quantified and compared to the control cells treated only with fAβ3488 (FIGS. 5A-5B). The fluorescence of fAβ3488 was markedly reduced by 70% and 60% in cells treated with NPs with bioactive shells (T₁₂P₅) (*p=0.0016) and (M₁₂P₅) (*p=0.0059) compared to control cells treated with fAβ488, respectively (FIG. 5B). This reduction in fAβ is presumably due to the putative binding ability of the bioactive shells to SRs on the microglial surface. This result was confirmed by the observation that the fluorescence of fAβ488 in cells treated with control NPs comprised of inactive shells (PS-b-PEG) was not appreciably altered compared to control cells treated only with fAβ488. Collectively, these results confirm the ability of AM-NPs to interrupt microglial fAβ uptake through combined effects on fAβ-specific SRs.

### Example 4. AM-NPs Modulate Microglial Inflammatory Response and Neurotoxicity

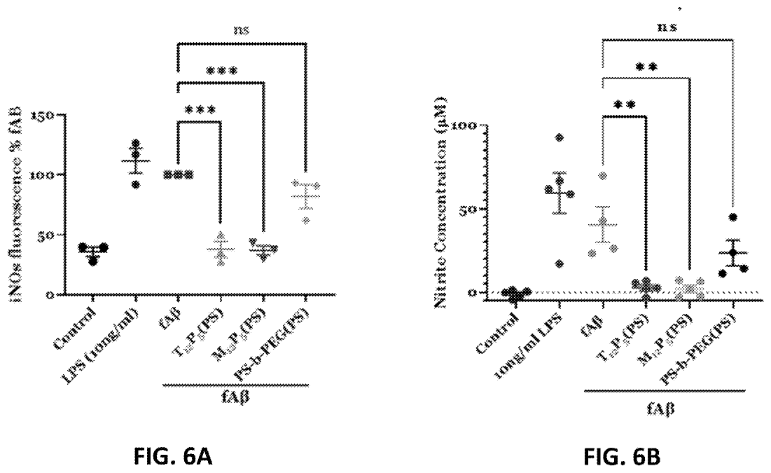

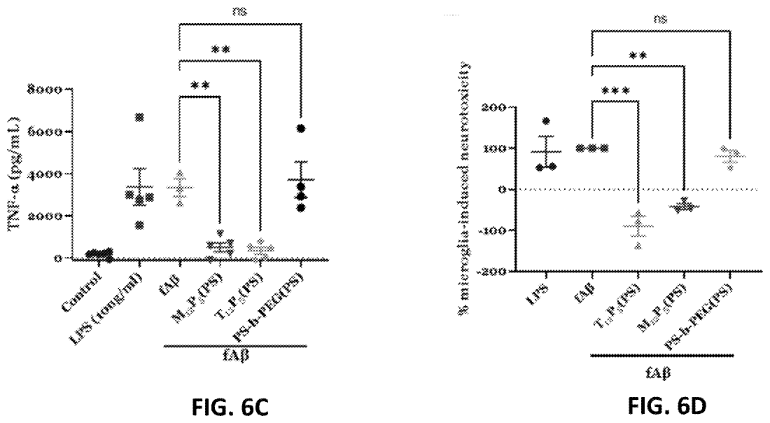

The recognition of fAβ by SRs on microglia results in a pro-inflammatory response characterized by the release of pro-inflammatory cytokines and chemokines. Excessive exposure to fAβ results in microglial activation and a shift from quiescent to a pro-inflammatory microglial phenotype. Therefore, the effect of AM-NPs on fAβ-mediated pro-inflammatory response in microglia was evaluated by assessing the cellular expression of inducible nitric oxide synthase (iNOS) and the level of released tumor necrosis factor-alpha (TNF-α) and nitrite (NO) (FIGS. 6A-6D). BV2 microglia treated with 20 μM fAβ were co-incubated with or without AM-NPs or with or 10 ng/ml lipopolysaccharide (LPS) for 24 h. After 24 h, supernatants were collected and assayed for inflammatory markers. Cells were fixed with 4% paraformaldehyde (PFA) then permeabilized in 0.5% triton-X-100 in potassium-buffered saline (PBS-T). After blocking, cells were incubated with anti-iNOS antibody overnight at 4° C., then washed with PBS-T. Cells were incubated with secondary antibody Alex 594 for 1 h. Cellular expression of iNOS was significantly reduced in fAβ-stimulated cells co-incubated with T₁₂P₅(PS) (p=0.0002) and M₁₂P₅(PS) (p=0.0001) compared to control cells treated with fAβ only (FIG. 6A). In line with this result, the concentration of NO released from fAβ-stimulated microglia co-incubated with T₁₂P₅(PS) (p=0.0057) or M₁₂P₅(PS) (p=0.0048) was significantly reduced compared to control cells treated with fAβ only (FIG. 6B). Similarly, the concentration of TNF-α was significantly reduced in fAβ-stimulated microglia co-incubated with T₁₂P₅(PS) (p=0.0042) or M₁₂P₅(PS) (p=0.0068) compared to control cells treated with fAβ only (FIG. 6C). Given the pronounced inflammatory response in fAβ-stimulated cells co-incubated with PS-b-PEG(PS) NPs, these results confirm the anti-inflammatory effects of the bioactive shells T₁₂P₅ and M₁₂P₅ on fAβ-mediated activation.

In AD, microglial activation and the subsequent release of pro-inflammatory cytokines lead to neuronal damage and loss of neuronal circuits (He, P et al. J Cell Biol 2007; 178(5):829-41 and Cagnin, A, et al. The Lancet;358(9280):461-7). Therefore, given the anti-inflammatory effects of AM shells described earlier, the role of AM-NPs in modulating microglia-mediated neurotoxicity was evaluated. To recapitulate the role of the cells in AD brain, a conditioned media approach was employed based on BV2 microglial and SH-SY5Y neuroblastoma cells. fAβ-stimulated BV2 cells were co-incubated with or without AM-NPs T₁₂P₅(PS), M₁₂P₅(PS), or PS-b-PEG(PS) for 24 h. In parallel, positive control cells were treated with LPS for 24 h. SH-SY5Y cells were then treated with the harvested BV2-conditioned media (CM) and incubated for 24 h. Treatments with T₁₂P₅(PS) and M₁₂P₅(PS) CM were significantly reduced neurotoxicity (p=0.003) and (p=0.0027), respectively (FIG. 6D). Consistent with the microglial activation studies, the non-bioactive shell in control NP PS-b-PEG(PS) didn't counteract the BV2-mediated neurotoxicity, which supports the notion that the bioactive shells can modulate active microglia-mediated neurotoxicity. In particular, NPs with T₁₂P₅ bioactive shells resulted in further reduced neurotoxicity compared to NPs with M₁₂P₅ bioactive shells (FIG. 6D).

The interaction of T₁₂P₅ with CD36 and M₁₂P₅ with CD36 and SRA1 receptors was previously validated (Zhao, N et al. Advanced Nanobiomed Research. 2022;2(6):2100120 and Plourde, N M et al. Biomacromolecules. 2009; 10(6):1381-91). These experiments expand the knowledge about T₁₂P₅ to include SRA1 and CD68 as receptor-specific macromolecules. Treatment with T₁₂P₅ alone was sufficient to interrupt the internalization of fAβ into microglia to an extent comparable to that elicited through the combined inhibitory effects of SRA1, DC36, and CD68 receptor-targeting antibodies (FIGS. 6A-6D). Accordingly, SRA1 deficiency caused 50% reduction in Aβ uptake by microglia in AD transgenic mice (Frenkel, D et al. Nat Commun. 2013;4(1):2030). Also, fAβ uptake by microglia isolated from SRA1 knockout mice was reduced by 60% while competed by other SRs ligands (Chung, H et al. Neuroreport. 2001; 12(6):1151-4). These results indicate that other SRs may be involved in the uptake of fAβ. Of these receptors is SR-B, CD36, which has been widely studied in fAβ trafficking and microglial activation (El, Khoury et al. Neurobiology of Aging. 1998;19(1, Supplement 1):S81-S4; Thanopoulou, K et al. PNAS. 2010; 107(48):20816-21; and Coraci, I S et al. The American Journal of Pathology. 2002; 160(1):101-12). Despite the findings that the M₁₂P₅ shells were shown to exert no competing effects with SR-specific antibodies, these molecules significantly interrupt fAβ uptake by microglia (FIGS. 6A-6D). In line with these findings, these M₁₂P₅ shells have been observed to bind SRA1 and CD36 (Plourde, N M et al. Biomacromolecules. 2009; 10(6):1381-91) and markedly interrupt the internalization of ASYN into microglia (Bennett, N K et al. Biomaterials. 2016; 111:179-89). Key differences between these AMs and SR antibodies include lipophilicity, charge, and binding sites on SRs. It is possible that M₁₂P₅ AMs block other intracellular epitopes on SRs that bind to and facilitate fAβ uptake. It is also possible that these AMs are able to prevent the formation of multi-receptor complexes that exert a signaling effect in the progression of fAβ pathogenesis.

It was reported that CD36 induces microglial pro-inflammatory signaling through heterodimer assembly with TLR2 and TLR4 (Stewart, C R et al. Nat Immunol. 2010; 11(2):155-61). Moreover, interrupting CD36 receptor complex with the integrin-associated protein CD47, and α6β1-integrin was found to prevent cells adhesion to fAβ and the subsequent pro-inflammatory response (Bamberger, M E et al. J Neurosci. 2003; 23(7):2665-74).

Consequently, AM-NPs interrupted fAβ-mediated activation, pro-inflammatory response, and neurotoxicity. The levels of iNOS, TNF-α, and NO were significantly reduced to the baseline levels in cells stimulated with fAβ3. Because SRA1 and CD36 were both shown to mediate microglial fAβ uptake and the consequent pro-inflammatory cytokines secretion (Khoury, J E et al. Nature. 1996; 382(6593):716-9 and Stewart, C R et al. Nat Immunol. 2010; 11(2):155-61), it is possible that the combined inhibitory effect of AM-NPs on multiple SRs would cause the observed anti-inflammatory effects in the presence of fAβ. It was shown that the secretion of ROS reduced by 50% in cells blocked with antibodies against CD36 and plated on fAβ-coated surfaces (Coraci, I S et al. The American Journal of Pathology. 2002; 160(1):101-12). Consistent with this observation, CD36 engagement by fAβ initiates a CD36-dependent signaling cascade, which leads to an inflammatory response, including the production of ROS and chemokines. The inhibition of this signaling cascade was found to significantly reduce ROS in the presence of CD36 and fAβ in vitro (Moore, K J et al. J Biol Chem. 2002; 277(49):47373-9). Similar results were observed in CD36 knockout macrophages stimulated by fAβ (El Khoury, J B et al. J Exp Med. 2003; 197(12):1657-66). These results indicated that SRs and the downstream signaling cascade are essential to fAβ-mediated inflammatory response. The AM-NPs were observed to have more of an effect than antibody treatments in reducing intracellular fibrils, the associated pro-inflammatory response, and neurotoxicity. Therefore, targeting multiple SRs using AM-NPs is a multi-faceted approach to interrupt fAβ uptake and the downstream signaling events essential for fAβ pathological progression.

### Example 5. AM-NPs Modulate NF-κB Nuclear Translocation and Induce Lysosomal Clearance

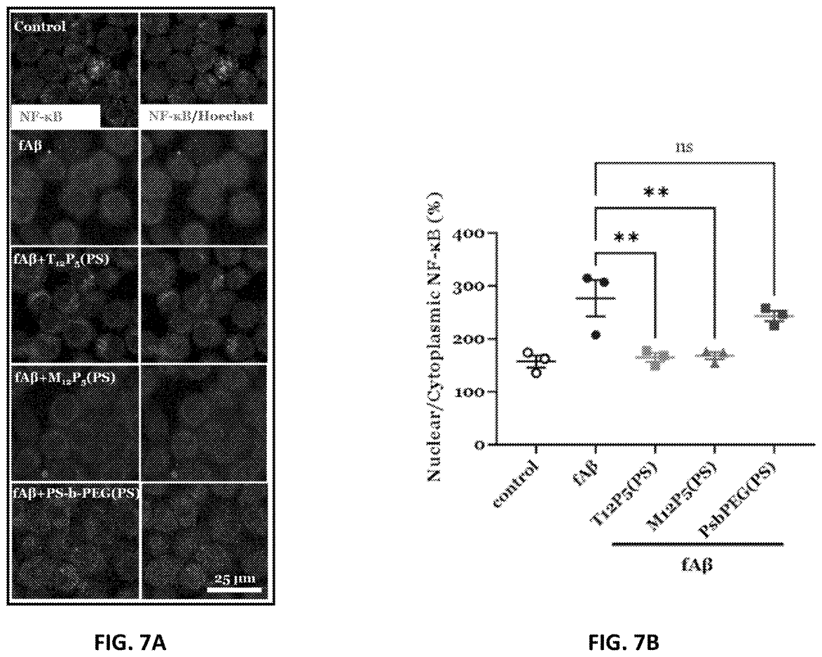

The production of inflammatory cytokines and chemokines is regulated by upstream events including the translocation of the cytoplasmic NF-κB into the nucleus. In quiescent microglia, NF-κB is sequestered in the cytoplasm; however, following a cellular stimulus such as fAβ, NF-κB translocated into the nucleus where it induces the expression of pro-inflammatory cytokines and chemokines genes (Thawkar, B S et al. J Neuroimmunol. 2019; 326:62-74 and Chen, J et al. J Biol Chem. 2005; 280(48):40364-74). Given the modulatory effects of AM-NPs on fAβ-mediated inflammation, it was determined whether these effects are due to a regulatory effect of NPs on an upstream event such as NF-κB nuclear translocation. fAβ-stimulated BV2 microglia were co-incubated with or without AM-NPs for 2 h, and then cells were immediately fixed with 4% PFA. Cells were then blocked using 2% blocking buffer, incubated with anti-NF-κB P65 antibody overnight at 4° C., and washed with PBS-T. Cells were incubated with secondary antibody Alex 488 for 1 h and nuclei were counterstained with Hoechst. Using immunocytochemistry and confocal microscopy, the immunoreactivity of the nuclear and the cytoplasmic NF-κB was determined in cells counterstained with Hoechst nuclear stain (FIGS. 7A-7B). Consistent with its pro-inflammatory effects, treatment with fAβ significantly increased the nuclear NF-κB (p<0.0027) (FIG. 7B). Strikingly, nuclear NF-κB was significantly reduced in fAβ-stimulated cells co-incubated with T₁₂P₅(PS) (p<0.0044) and M₁₂P₅(PS) (p<0.0054) which attenuates the fAβ-induced inflammatory response.

The nuclear factor-κB (NF-κB) is a well-established transcription factor that plays a central role in mediating the inflammatory response in AD (Sun, E et al. Int J Mol Sci. 2022;23(16)). Under physiological conditions, NF-κB is sequestered in the cytoplasm; however, upon exposure to pro-inflammatory stimuli, it translocates into the nucleus. Nuclear translocation of NF-κB activates the expression of pro-inflammatory cytokines and chemokines (Chiarini, A. et al. Int J Mol Sci. 2020;21(23):9036). The results showed that T₁₂P₅(PS) and M₁₂P₅(PS) could significantly reduce fAβ-induced nuclear translocation of NF-κB (FIGS. 7A-7B). This finding indicates that the mechanism through which AM-NPs modulate fAβ-induced microglial activation, and the pro-inflammatory response may primarily be the NF-κB signaling pathway. Since the extensive release of pro-inflammatory cytokines can activate the NF-κB signaling pathway, it is possible that AM-NPs potentially interrupt this vicious cycle of fAβ-mediated microglial activation. Molecules that targeted the NF-κB signaling pathway have been shown to decrease inflammatory response, improve behavioral deficits, and promote neuroprotection in AD models (Cai, Z et al. Pharmacol Rep. 2011; 63(2):381-91; Gagliardi, S et al. Acta Neuropathol. 2018; 9:1404; and Chen, J et al. J Biol Chem. 2005; 280(48):40364-74). NF-κB signaling components were identified as top upstream regulators in tau-stimulated microglia. For example, inhibition of the NF-κB pathway by TPCA-1 was found to curtail phosphorylated tau released from microglia and rescue tau-associated learning and memory deficits (Wang, C et al. Nat Commun. 2022;13(1):1969). AM-NPs could lead to the development of a mechanism-based therapeutic to target NF-κB translocation in multiple cell types and arrest microglial activation in several neurological disorders.

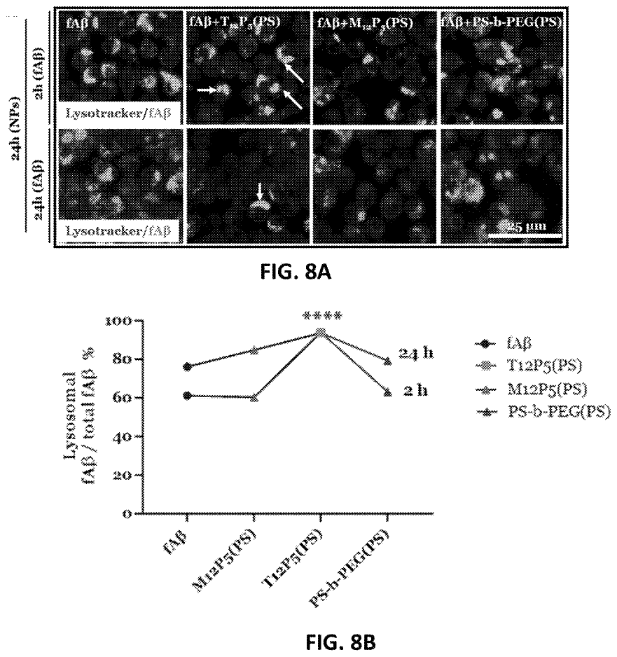

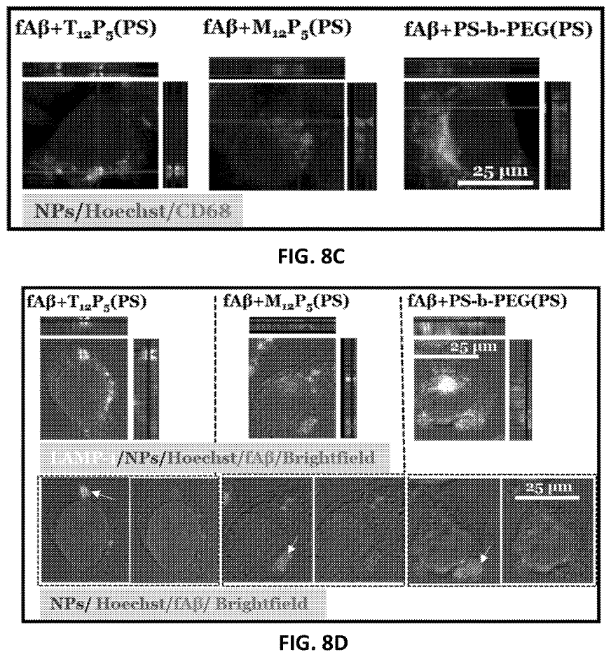

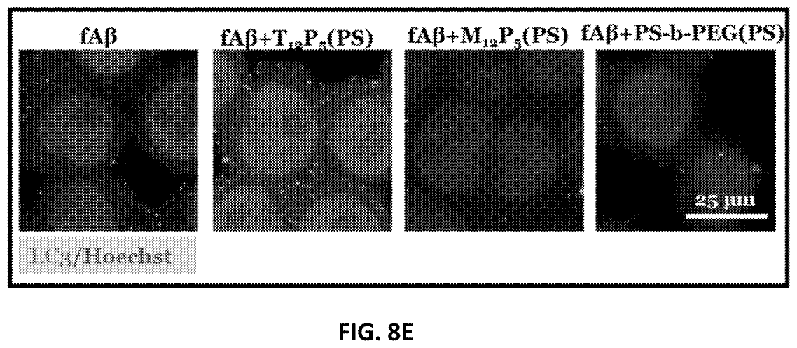

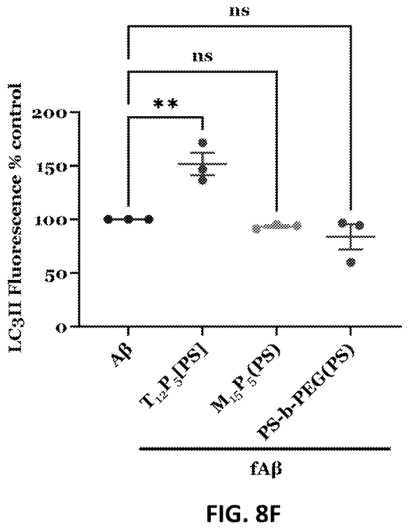

Chronic activation of microglia due to prolonged exposure to fAβ results in loss of homeostatic microglial function and imbalanced clearance of fAβ. Dysregulated clearance of fAβ, which has been observed in brains of AD patients and animal models (Hickman, S E et al. J Neurosci. 2008; 28(33):8354-60; Nixon RA. FASEB J. 2017;31(7):2729; and Cataldo, A M et al. The American Journal of Pathology. 2008; 173(2):370-84), exacerbates fAβ burden and leads ultimately to neuronal damage. Because the data revealed modulatory effects of AM-NPs on early events of the fAβ pathway, including amyloid beta fibrilization, cellular uptake, NF-κB nuclear translocation, and microglial activation; the possible role of NPs on the lysosomal functional processes related to clearance in microglia was examined. BV2 microglia were co-incubated with fAβ for either 2 h or 24 h after pre-incubation for 24 h with or without AM-NPs. fAβ in cells treated with T₁₂P₅(PS) were significantly (p<0.0001) localized in lysosomes compared to control cells treated with fAβ only (FIGS. 8A-8B). While fAβ in cells co-treated with the control NPs didn't show any change from the control cells treated with fAβ only. To further validate the observation that T₁₂P₅ accelerated degradation of fAβ at 2 h, the immunoreactivity of the lysosomal-associated protein (LAMP-1) and CD68 were assayed. Using colocalization approaches, the internalized fluorescently labeled T₁₂P₅ (PS) demonstrated a high degree of colocalization with fAβ in lysosomes as evidenced by colocalization with LAMP-1 (FIG. 8C). These results were confirmed by the observation that the fluorescently labeled T₁₂P₅(PS) and fAβ488 were shown to be colocalized with the lysosomal receptor CD68 (FIG. 8D). Interestingly, in cells treated with M₁₂P₅(PS), fAβ488 was found in association with the fluorescently labeled NPs; however, these molecules were not in association with LAMP-1. On the other hand, in cells treated with control NPs, fluorescently labeled NPs were diffused in most areas of the cytoplasm, while fAβ488 was observed in LAMP-1 negative compartment in the cytoplasm (FIG. 8C), indicating the inefficient cellular ability to affect clearance of fAβ deposits. The observation that T₁₂P₅(PS) was colocalized with the lysosomal receptor CD68 (FIG. 8D) confirmed the hypothesis that these NPs may accelerate fAβ clearance through specific binding and recognition by CD68 which may act as cargo molecules between the cytoplasm and the lysosome (Chistiakov, D A et al. Laboratory Investigation. 2017; 97(1):4-13). These results suggest another unique feature of the tartaric acid-based shells, possibly consistent with their interactions with lysosomal receptor CD68 as described earlier.

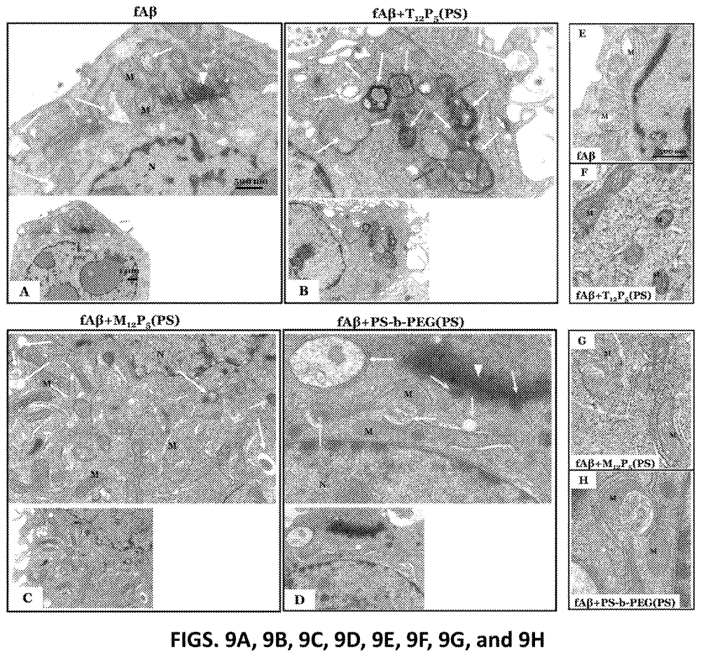

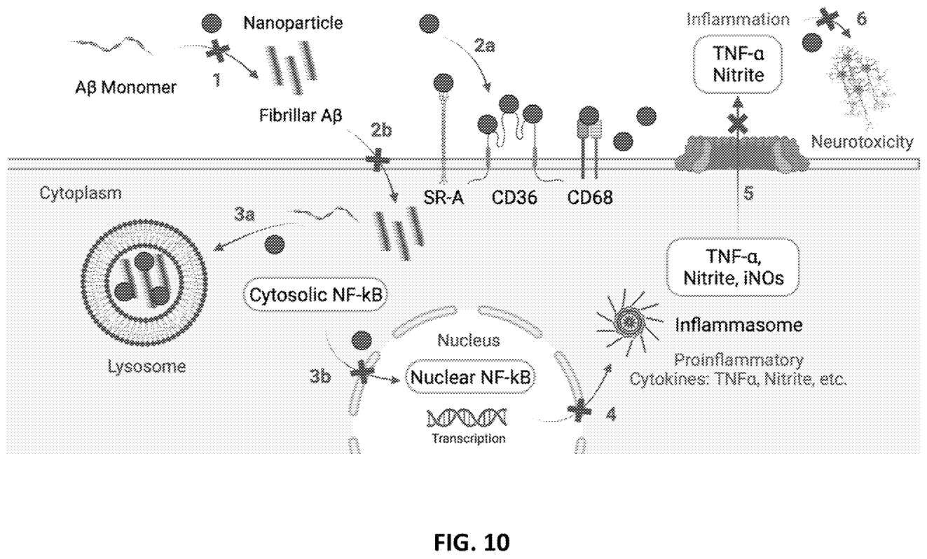

Additionally, it was found that cells in which fAβ-treated microglia were co-incubated with T₁₂P₅(PS) exhibited high phagocytic activity (FIG. 9B). Cells in which fAβ-treated microglia were co-incubated with M₁₂P₅(PS) exhibited less phagocytic activity as evidenced by fewer autophagosomes (FIG. 9F). Collectively, electron micrographs of BV2 microglia incubated with NPs and fAβ illustrated that the AM-NPs accelerate clearance of fAβ (FIGS. 10A-10H).

It is possible that the specific interactions of T₁₂P₅ with the lysosomal CD68 (FIGS. 3A-3D) and their selective binding with fAβ fragments (FIGS. 2A-2D) would lead to the formation of fAβ-T₁₂P₅-CD68 complex. Therefore, the fAβ-T₁₂P₅-CD68 complex may facilitate the transfer of fAβ into lysosomes and accelerate their clearance (FIG. 8C). Also, the interactions of fAβ with T₁₂P₅ may result in the formation of an fAβ-T₁₂P₅ complex that can be easily recognized by lysosomes as evident by the association of the lysosomal intracellular vesicle LAMP-1 and the fluorescently labeled T₁₂P₅(PS) (FIG. 8D). Few reports have highlighted the role of SRs in Aβ phagocytic clearance (Frenkel, D et al. Nat Commun. 2013;4(1):2030; Han, B H et al. Int J Mol Sci. 2022;23(11):5885; and Nakamura, K et al. J Neurosci Res. 2006; 84(4):874-90). However, one study suggested that SRA1 specifically participates in fAβ clearance, but does not play a rate-limiting role in fAβ clearance (Huang, F et al. The American Journal of Pathology. 1999; 155(5):1741-7). fAβ binding via immune receptors have been shown to be insufficient to trigger degradation of internalized fAβ in primary microglia (Brazil, M I et al. J Biol Chem. 2000; 275(22):16941-7), while microglia in brain slices could efficiently digest some plaques (Bard, F et al. Nat Med. 2000; 6(8):916-9). Consistent with this notion, immunotherapy has been shown efficient in reducing fAβ deposits in APP transgenic mice lacking the key immune receptors, Fc gamma receptors (FcRγ) (Das, P et al. J Neurosci 2003; 23(24):8532-8). These findings support the involvement of other receptors and mechanisms in the digestion of intracellular fAβ deposits. See FIG. 10 for a depiction of how AM-NPs interrupt amyloid beta fibrilization and affect cellular trafficking and fAβ-mediated neuronal damage.

To date, passive immunotherapeutic approaches, including several anti-Ap antibodies, have been investigated for their effects on targeting numerous forms of Aβ (Lee, E B et al. Journal of Biological Chemistry. 2006; 281(7):4292-9; Poduslo, J F et al. J Neurochem. 2007; 102(2):420-33; and Moreth, J et al. Immunity & Ageing. 2013; 10(1):1-9). Despite the reported success in targeting toxic Aβ plaques and improved cognitive functions, most of these therapies were abandoned during clinical trials due to lack of efficacy or severe side effects (Madav, Y et al. Brain Research Bulletin. 2019; 146:171-84). Consequently, the nanoparticles described herein may serve as a therapeutic for AD.

### Example 6. In Vivo Testing of AM-NPs

Various studies were performed to evaluate AM-NPs in vivo.

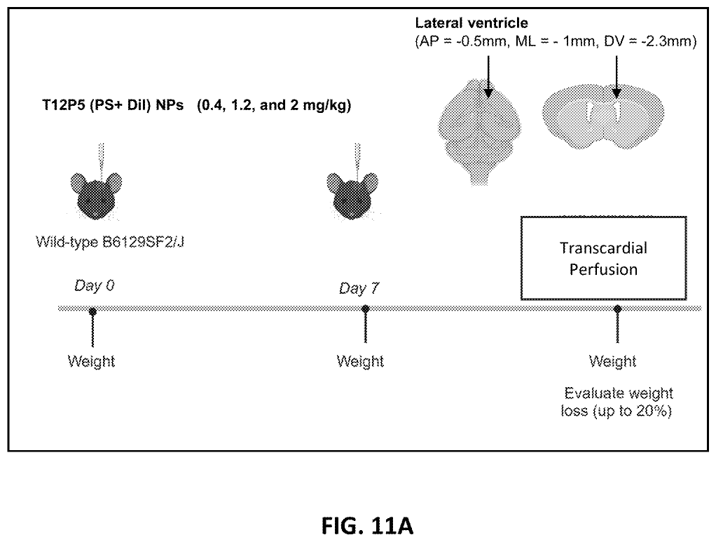

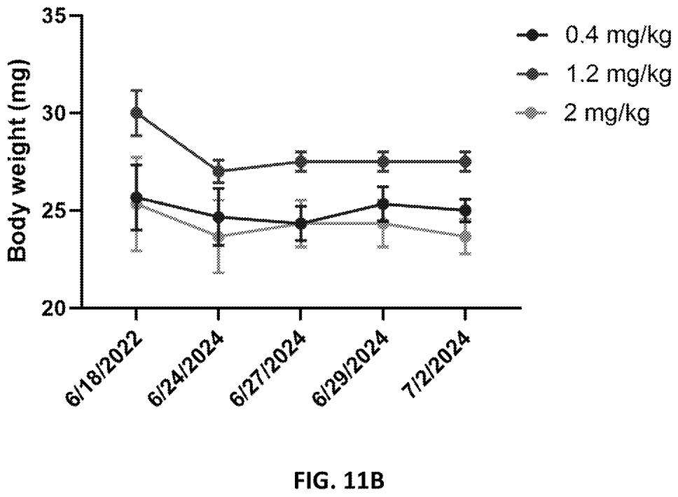

A study was conducted in mice to identify the maximum tolerated dose of AM-NPs. FIG. 11A summarizes the study design in wild-type B6129SF2/J mice administered 0.4 mg/kg, 1.2 mg/kg, or 2 mg/kg of T₁₂P₅(PS). Most mice injected with the varying doses of T₁₂P₅(PS) survived and experienced normal weight loss (FIG. 11B). Additionally, it was found that Dil-labeled AM-NPs diffused into the brain parenchyma in a dose-dependent manner without any adverse effects on the mice's general health.

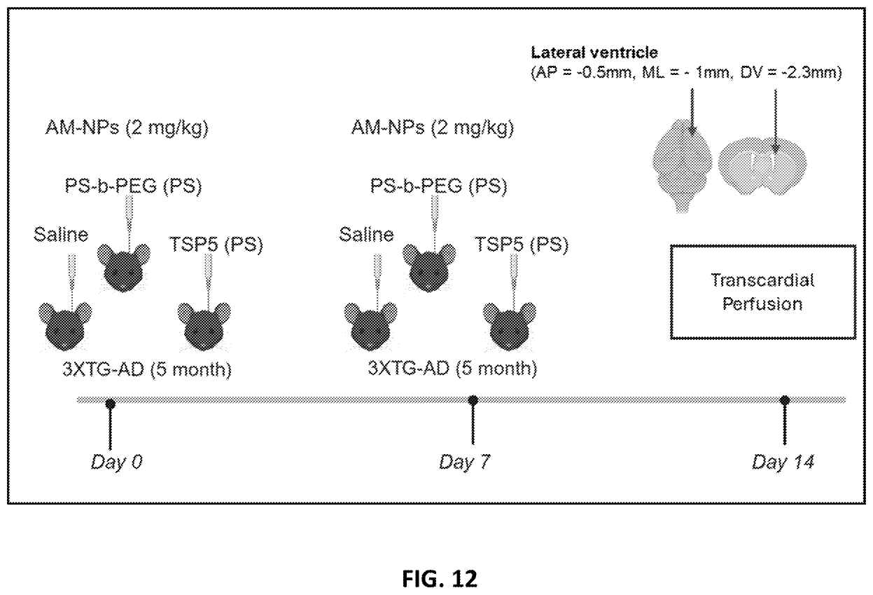

Another study was conducted to determine the efficacy of AM-NPs in 3XTG-AD mice (FIG. 12). These mice display both plaque and tangle pathology. Aβ deposition is progressive, with intracellular immunoreactivity detected in some brain regions as early as three to four months of age. Extracellular Aβ deposits appear by six months in the frontal cortex and become more extensive by twelve months (ALZFORM & Jackson lab). In this study, mice treated with T₁₂P₅ exhibited reduced fAβ levels and reduced neuroinflammation.

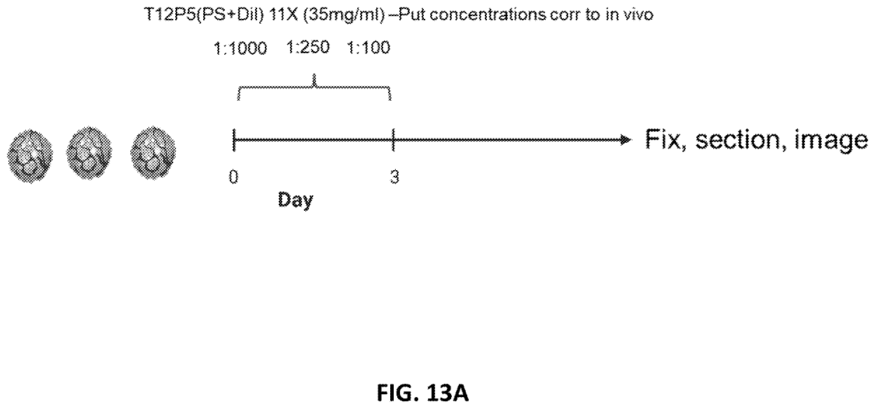

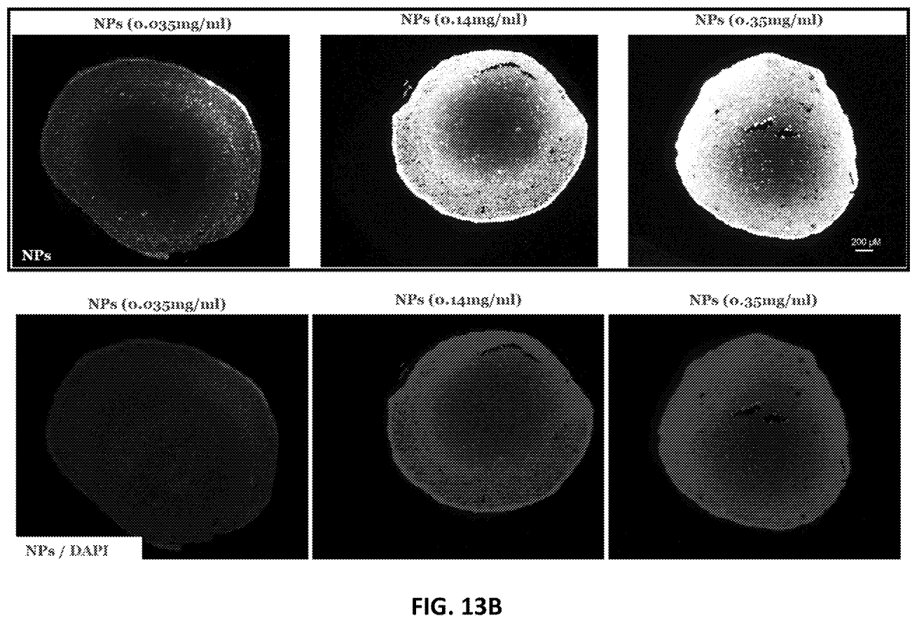

It was also found that T₁₂P₅ (PS+Dil) penetrates human cerebral organoids (hmCOs). An overview of the method used to obtain images of such organoids treated with T₁₂P₅(PS) is shown in FIG. 13A. Notably, Dil-labeled T₁₂P₅(PS) penetrates hmCOs in a dose-dependent manner. Images of hmCOs treated with 0.035 mg/ml, 0.14 mg/ml, or 0.35 mg/ml T₁₂P₅(PS) are shown in FIG. 13B.

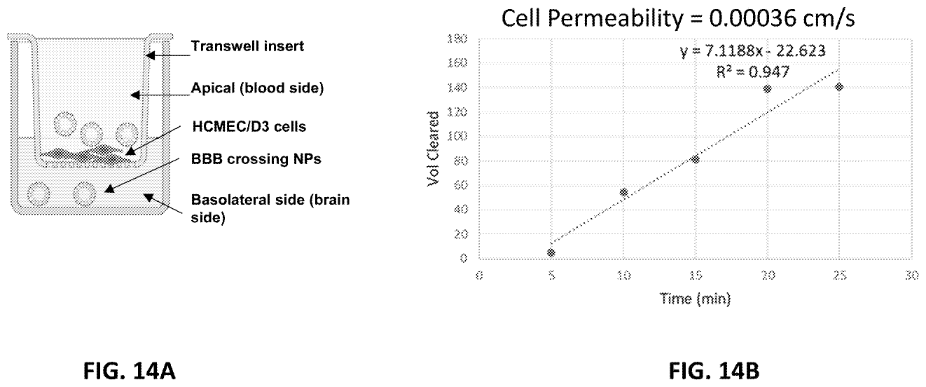

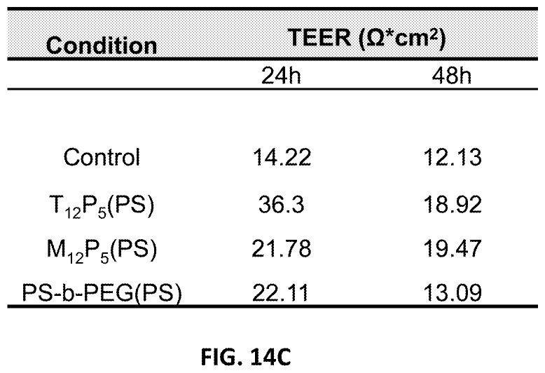

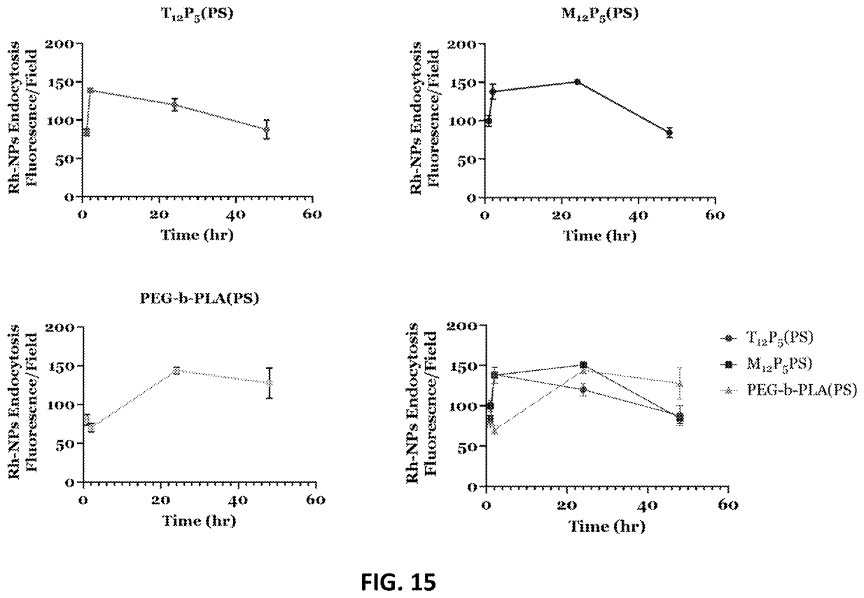

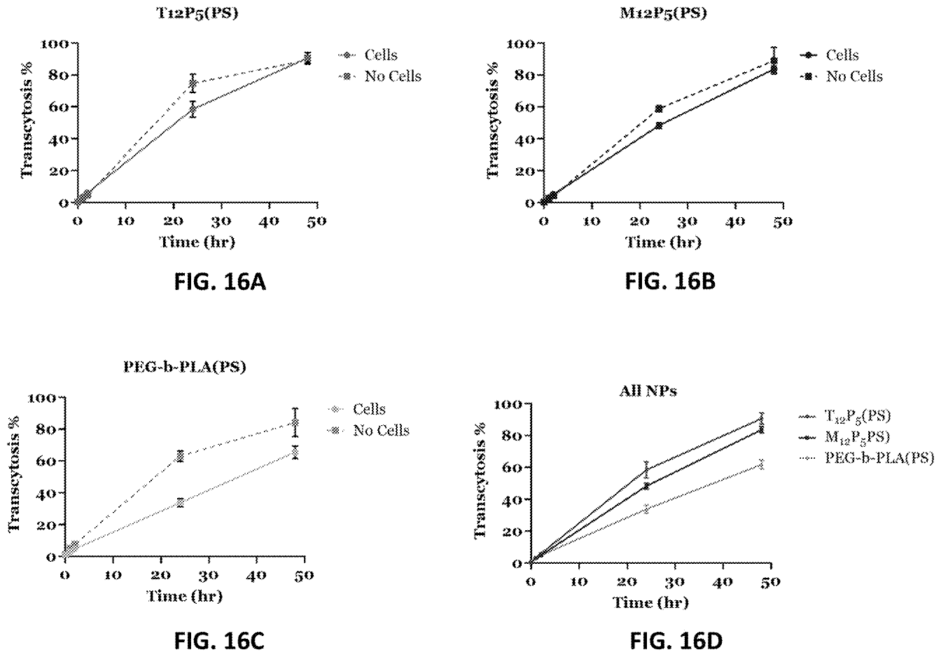

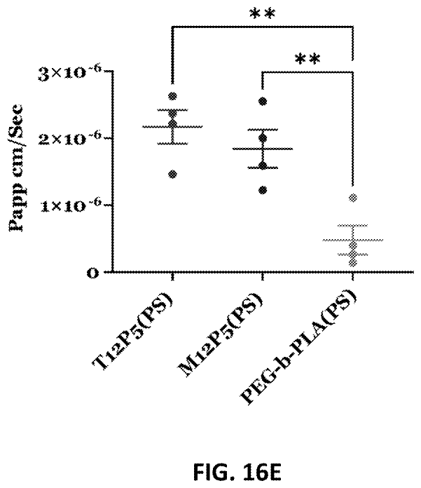

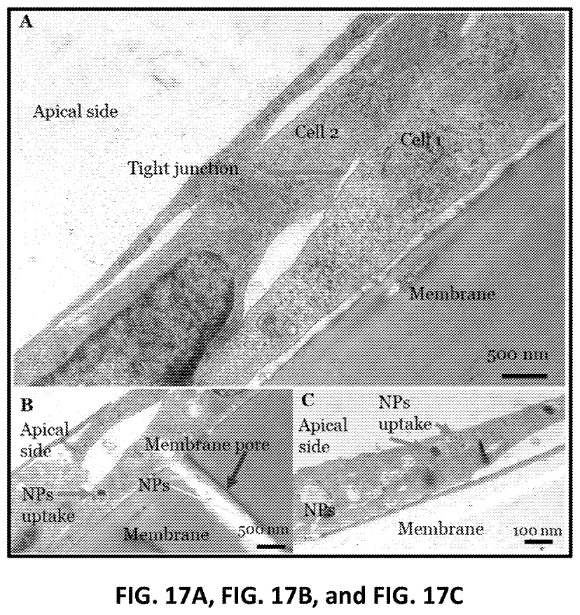

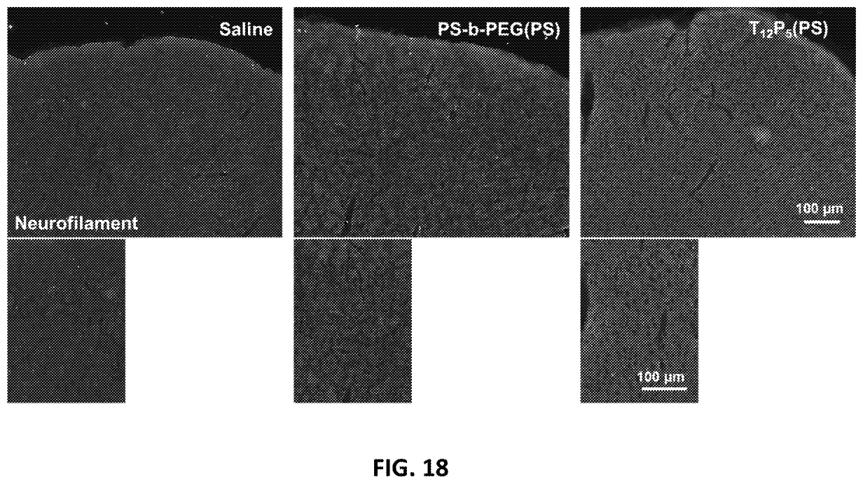

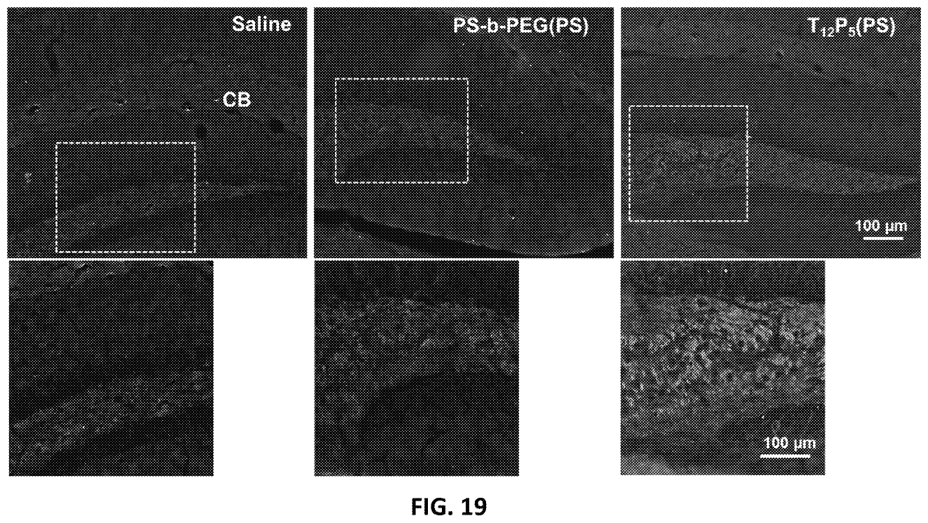

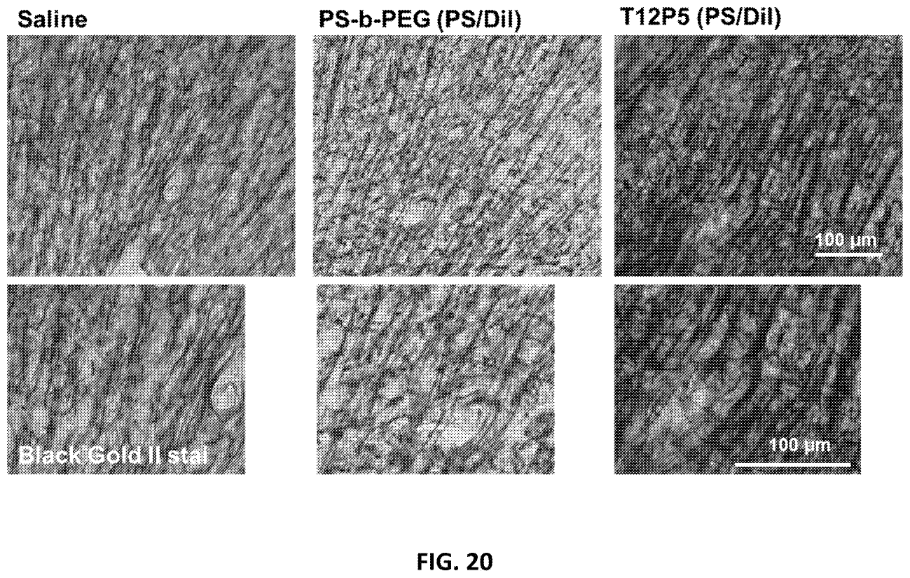

Lastly, a study was conducted in which hCMEC/D3 cells were treated with T₁₂P₅(PS). After validating the hCMEC/D3 monolayer cell model of blood brain barrier (BBB), Dil-labeled T₁₂P₅(PS) showed extensive endocytosis into the human cerebral endothelial cells (FIGS. 14A-14C).

What is claimed is:

1. A method of inhibiting amyloid beta (Aβ) fibrilization in a subject in need thereof, comprising administering a nanoparticle to the subject, wherein the nanoparticle comprises a hydrophobic core and a bioactive amphiphilic macromolecule that forms a shell coating the hydrophobic core, and
wherein the bioactive amphiphilic macromolecule comprises a structure set forth in Formula A or Formula B: 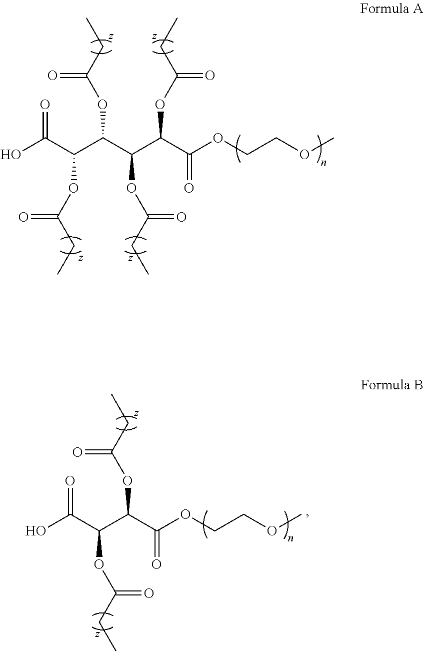
wherein n is an integer from 100 to 125; and
wherein z is an integer from 2 to 20.

2. The method of claim 1, wherein z is an integer from 4 to 12.

3. The method of claim 1, wherein n is an integer from 110 to 120.

4. The method of claim 1, wherein the diameter of the nanoparticle ranges from 90 nm to 200 nm.

5. The method of claim 1, wherein the polydispersity index (PDI) of the nanoparticle is less than 0.3.

6. The method of claim 1, wherein the bioactive amphiphilic macromolecule to hydrophobic core ratio by weight is 8:2.

7. The method of claim 1, wherein the nanoparticle binds to a fibril β-amyloid (fAβ)-specific scavenger receptor.

8. The method of claim 7, wherein the fAβ-specific scavenger receptor comprises CD36, CD68, or SRA1.

9. The method of claim 1, wherein the hydrophobic core comprises a structure set forth in Formula C: 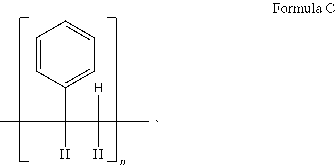
wherein n is an integer from 10 to 120.

10. The method of claim 1, wherein the subject is a human.

11. A method of treating Alzheimer's disease (AD) in a subject in need thereof, comprising administering a nanoparticle to the subject, wherein the nanoparticle comprises a hydrophobic core and a bioactive amphiphilic macromolecule coating the core, and wherein the bioactive amphiphilic macromolecule comprises a structure set forth in Formula A or Formula B: 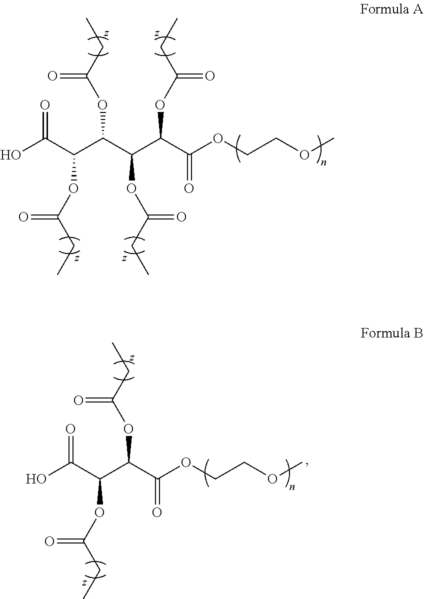
wherein n is an integer from 100 to 125; and
wherein z is an integer from 2 to 20.

12. The method of claim 11, wherein z is an integer from 4 to 12.

13. The method of claim 11, wherein n is an integer from 110 to 120.

14. The method of claim 11, wherein the diameter of the nanoparticle ranges from 90 nm to 200 nm.

15. The method of claim 11, wherein the polydispersity index (PDI) of the nanoparticle is less than 0.3.

16. The method of claim 11, wherein the bioactive amphiphilic macromolecule to hydrophobic core ratio by weight is 8:2.

17. The method of claim 11, wherein the nanoparticle binds to a fibril β-amyloid (fAβ)-specific scavenger receptor.

18. The method of claim 17, wherein the fAβ-specific scavenger receptor comprises CD36, CD68, or SRA1.

19. The method of claim 11, wherein the hydrophobic core comprises a structure set forth in Formula C: 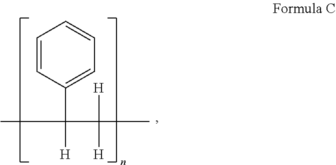
wherein n is an integer from 10 to 120.

20. A nanoparticle comprising a hydrophobic core and a bioactive amphiphilic macromolecule coating the core, and
wherein the bioactive amphiphilic macromolecule comprises a structure set forth in Formula A or Formula B: 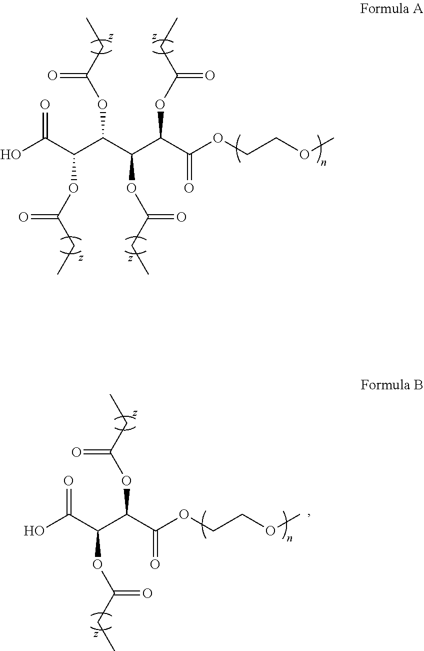
wherein n is an integer from 100 to 125; and
wherein z is an integer from 2 to 20.
